(19) United States
(12) Patent Application Publication
(10) Pub. No.: US 2026/0207203 A1
(43) Pub. Date: Jul. 23, 2026

(54) CLIP INSTRUMENTS AND CONTROL METHODS THEREOF

(71) Applicant: HANGZHOU AGS MEDTECH CO., LTD., Hangzhou (CN)

(72) Inventors: Feng YAO, Hangzhou (CN); Chao YAN, Hangzhou (CN); Jiaqi LI, Hangzhou (CN)

(73) Assignee: HANGZHOU AGS MEDTECH CO., LTD., Hangzhou (CN)

(21) Appl. No.: 19/568,717

(22) Filed: Mar. 16, 2026

Related U.S. Application Data
(63) Continuation of application No. PCT/CN2024/118494, filed on Sep. 12, 2024.

(30) Foreign Application Priority Data
Sep. 14, 2023 (CN) 202311189848.9

(51) Int. Cl. A61B 17/128; A61B 17/00; A61B 17/064; A61B 17/122; A61B 90/00

(52) U.S. Cl. CPC A61B 17/1285; A61B 17/0644; A61B 17/122; A61B 2017/00323; A61B 2090/035

(57) ABSTRACT

Provided are a clip instrument and a control method. The clip instrument includes a clip device, a delivery device, and a control device. The clip device is releasably disposed at a distal end of the delivery device. The control device includes a control wire including first and second control wires. The clip device includes a clip base and a clamping portion including a first clamping portion and a second clamping portion. A distal end of the first control wire is releasably connected to the first clamping portion. A distal end of the second control wire is releasably connected to the second clamping portion. The first and second clamping portions are configured to deflect in the same direction around an axis under an action of the first and/or the second control wire. The axis is perpendicular to a plane where the first and second clamping portions are located.

### CROSS-REFERENCE TO RELATED APPLICATIONS

This application is a Continuation of International Application No. PCT/CN2024/118494, filed on Sep. 12, 2024, which claims priority to the Chinese Patent Application No. 202311189848.9, filed on Sep. 14, 2023, the contents of each of which are hereby incorporated by reference.

### TECHNICAL FIELD

The present disclosure generally relates to a field of medical instruments, and in particular, to a clip instrument and a control method thereof.

### BACKGROUND

In recent years, endoscopic technology has developed rapidly, and various surgeries performed with the aid of the endoscope have emerged. In endoscopic surgery, a clip device is required to close the wound for hemostasis. In clinical operations, when the distal end of the endoscope reaches the vicinity of the wound, the clamping direction of the clip device may deviate from the opening direction of the wound. Therefore, the clip device needs to be adjusted to face the wound directly to achieve effective clamping. Typically, the clamping direction of the clip device is changed by adjusting the distal swing of the endoscope (or the sheath). However, it is difficult to operate and inaccurate in positioning for this way, leading to prolonged surgery time and increased surgical risk. In addition, existing clip devices typically do not have sufficient clamping force to clamp large-span tissue.

Based on this, a clip instrument with stable clamping is needed, which can more conveniently and accurately locate a position directly facing the wound and can clamp the large-span tissue, thereby simplifying surgical operations and reducing surgical risk.

### SUMMARY

One or more embodiments of the present disclosure provide a clip instrument. The clip instrument includes a clip device, a delivery device, and a control device. The clip device is releasably disposed at a distal end of the delivery device. The control device includes a control wire. The control wire includes a first control wire and a second control wire. The clip device includes a clip base and a clamping portion. The clamping portion includes a first clamping portion and a second clamping portion. A distal end of the first control wire is releasably connected to the first clamping portion. A distal end of the second control wire is releasably connected to the second clamping portion. The first clamping portion and the second clamping portion are configured to deflect in the same direction around an axis under an action of the first control wire and/or the second control wire. The axis is perpendicular to a plane in which the first clamping portion and the second clamping portion are located.

One or more embodiments of the present disclosure provide a clip instrument. The clip instrument includes a clip device. The clip device includes a clip base and a clamping portion. The clamping portion includes a first clamping portion and a second clamping portion. At least one of the first clamping portion or the second clamping portion includes a puncture member. The puncture member includes a puncture rod and a puncture head connected to each other. The puncture head includes a hooking portion facing the puncture rod.

One or more embodiments of the present disclosure provide a control method for a clip instrument. The control method is applied to the clip instrument. The clip instrument includes a clip device, a delivery device, and a control device. The clip device is releasably disposed at a distal end of the delivery device. The control device includes a control wire. The control wire includes a first control wire and a second control wire. The clip device includes a clip base and a clamping portion. The clamping portion includes a first clamping portion and a second clamping portion. A distal end of the first control wire is releasably connected to the first clamping portion. A distal end of the second control wire is releasably connected to the second clamping portion. The control method includes: controlling the first control wire to move from a proximal end to a distal end of the first clamping portion to cause the first control wire to drive the first clamping portion to deflect around an axis; and controlling the second control wire to move from a distal end to a proximal end of the second clamping portion to cause the second control wire to drive the second clamping portion to deflect around the axis. A deflection direction of the first clamping portion and a deflection direction of the second clamping portion are the same, and the clip device deflects integrally.

One or more embodiments of the present disclosure provide a control method for a clip instrument. The control method is applied to the clip instrument. The clip instrument includes a clip device, a delivery device, and a control device. The clip device is releasably disposed at a distal end of the delivery device. The control device includes a control wire. The control wire includes a first control wire and a second control wire. The clip device includes a clip base and a clamping portion. The clamping portion includes a first clamping portion and a second clamping portion. A distal end of the first control wire is releasably connected to the first clamping portion. A distal end of the second control wire is releasably connected to the second clamping portion. The control method includes: controlling the first control wire to move toward a distal end to cause the clip instrument to be in a side-open state; and when a side-opening angle of the clip instrument reaches a maximum value, continuing to control the first control wire to move toward the distal end, wherein the first clamping portion and the second clamping portion deflect in the same direction.

One or more embodiments of the present disclosure provide a control method for a clip instrument. The control method is applied to the clip instrument. The clip instrument includes a clip device, a delivery device, and a control device. The clip device is releasably disposed at a distal end of the delivery device. The control device includes a control wire. The control wire includes a first control wire and a second control wire. The clip device includes a clip base and a clamping portion. The clamping portion includes a first clamping portion and a second clamping portion. A distal end of the first control wire is releasably connected to the first clamping portion. A distal end of the second control wire is releasably connected to the second clamping portion. At least one of the first clamping portion or the second clamping portion includes a puncture member. The control method includes: controlling the clip instrument to clamp a first target object at a first target position and causing the puncture member to penetrate into the first target object; controlling at least a portion of the clip device to move to a second target position, the puncture member driving the first target object to move synchronously; and controlling the clip instrument to simultaneously clamp the first target object and a second target object.

### BRIEF DESCRIPTION OF THE DRAWINGS

The present disclosure will be further illustrated by way of exemplary embodiments. The exemplary embodiments will be described in detail with reference to the drawings. The embodiments are not limiting. In the embodiments, the same reference numerals denote the same structures, wherein:

FIG. 1 is a schematic structural diagram of a clip instrument according to some embodiments of the present disclosure;

FIG. 2 is a schematic structural diagram of a clip device according to some embodiments of the present disclosure;

FIG. 3 is a schematic structural diagram of a clamping portion according to some embodiments of the present disclosure;

FIG. 4 is a schematic structural diagram of a clip base according to some embodiments of the present disclosure;

FIG. 5A is a schematic diagram of a clip device and a delivery device according to some embodiments of the present disclosure;

FIG. 5B is an exploded schematic diagram of a clip device and a delivery device according to some embodiments of the present disclosure;

FIG. 6 is a schematic structural diagram of a control wire according to some embodiments of the present disclosure;

FIG. 7 is a schematic diagram of deflection of a clip instrument according to some embodiments of the present disclosure;

FIG. 8A is a schematic diagram of deflection of a clip instrument according to some embodiments of the present disclosure;

FIG. 8B is a partially enlarged schematic diagram of the clip instrument according to some embodiments of FIG. 8A;

FIG. 8C is a partially enlarged schematic diagram of the clip instrument according to other embodiments of FIG. 8A;

FIG. 8D is a first simplified cross-sectional diagram of the clip instrument taken along a line C-C according to some embodiments of FIG. 8A;

FIG. 8E is a second simplified cross-sectional diagram of the clip instrument taken along the line C-C according to some embodiments of FIG. 8A;

FIG. 9 is a schematic diagram of a control device according to some embodiments of the present disclosure;

FIG. 10 is a schematic diagram of a control device according to some embodiments of the present disclosure;

FIG. 11 is a partially enlarged schematic diagram of a clip instrument according to some embodiments of the present disclosure;

FIG. 12 is a schematic structural diagram of a connection pin according to some embodiments of the present disclosure;

FIG. 13 is a schematic diagram of an open state of a clip instrument according to some embodiments of the present disclosure;

FIG. 14 is a schematic diagram of a deflected state of a clip instrument according to some embodiments of the present disclosure;

FIG. 15 is a schematic diagram of a deflected state of a clip instrument according to other embodiments of the present disclosure;

FIG. 16A is a schematic diagram of a closed state of a clip instrument according to some embodiments of the present disclosure;

FIG. 16B is a partially enlarged schematic diagram of the clip instrument according to some embodiments of FIG. 16A;

FIG. 17A is a schematic diagram of a locked state of a clip instrument and released states of a clamping portion and a control wire according to some embodiments of the present disclosure;

FIG. 17B is a partially enlarged schematic diagram of the clip instrument according to some embodiments of FIG. 17A;

FIG. 18A is a schematic diagram of a released state of a clip device and a delivery device according to some embodiments of the present disclosure;

FIG. 18B is a partially enlarged schematic diagram of the clip instrument according to some embodiments of FIG. 18A;

FIG. 19A is a schematic structural diagram of a first clamping portion according to some embodiments of the present disclosure;

FIG. 19B is a schematic structural diagram of a second clamping portion according to some embodiments of the present disclosure;

FIG. 19C is a schematic diagram of a closed state of a clip device according to some embodiments of the present disclosure;

FIG. 19D is a partially enlarged schematic diagram of the clip device according to some embodiments of FIG. 19C;

FIG. 19E is a schematic diagram of a released state of a clip device according to some embodiments of the present disclosure;

FIG. 19F is a partially enlarged schematic diagram of the clip device according to some embodiments of FIG. 19E;

FIG. 20 is a schematic structural diagram of a clip device according to some other embodiments of the present disclosure;

FIG. 21A is a schematic structural diagram of a puncture member according to some embodiments of the present disclosure;

FIG. 21B is a schematic structural diagram of a puncture member according to some other embodiments of the present disclosure;

FIG. 21C is a schematic structural diagram of the puncture member according to other embodiments of the present disclosure;

FIG. 21D is a schematic structural diagram of a puncture member according to yet other embodiments of the present disclosure;

FIG. 22 is a schematic diagram of a height relationship between a puncture member and a first clip head according to some embodiments of the present disclosure;

FIG. 23 is a schematic structural diagram of a puncture platform according to some embodiments of the present disclosure;

FIG. 24 is a schematic diagram of height relationships between a puncture member and a first clip head and between a puncture platform and a second clip head according to some embodiments of the present disclosure;

FIG. 25 is a schematic diagram of a length relationship between a puncture member and a clamping portion according to some embodiments of the present disclosure;

FIG. 26 is a schematic diagram of an open state of a clip instrument according to some other embodiments of the present disclosure;

FIG. 27A is a schematic diagram of a deflected state of a clip instrument according to some other embodiments of the present disclosure;

FIG. 27B is a schematic diagram of a control handle in the deflected state according to some embodiments of FIG. 27A;

FIG. 27C is a schematic diagram of a control handle in the deflected state according to some other embodiments of FIG. 27A;

FIG. 28A is a schematic diagram of a clip instrument clamping tissue after side-opening according to some embodiments of the present disclosure;

FIG. 28B is a schematic diagram of a control handle in a state according to some embodiments of FIG. 28A;

FIG. 29A is a schematic diagram of a secondary open state of a clip instrument according to some embodiments of the present disclosure;

FIG. 29B is a schematic diagram of a control handle in a state according to some embodiments of FIG. 29A;

FIG. 29C is a schematic diagram of a secondary open state of a clip instrument according to some other embodiments of the present disclosure;

FIG. 30A is a schematic diagram of a secondary closed state of a clip instrument according to some embodiments of the present disclosure;

FIG. 30B is a schematic diagram of a control handle in a state according to some embodiments of FIG. 30A;

FIG. 31A is a schematic diagram of a released state of a clip device and a delivery device according to some other embodiments of the present disclosure;

FIG. 31B is a schematic diagram of a control handle in a state according to some embodiments of FIG. 31A.

### DETAILED DESCRIPTION

To illustrate the technical solutions in the embodiments of the present disclosure more clearly, the following briefly introduces the drawings required for describing the embodiments. Obviously, the drawings in the following description show only some examples or embodiments of the present disclosure. For a person of ordinary skill in the art, the present disclosure may be applied to other similar scenarios according to these drawings without creative efforts. Unless obviously obtained from the context or the context illustrates otherwise, the same numeral in the drawings refers to the same structure or operation.

It should be understood that the terms “system,” “device,” “unit,” and/or “module” used herein are ways for distinguishing different components, elements, parts, sections, or assemblies at different levels. However, if other words can achieve the same purpose, the words may be replaced by other expressions.

As shown in the present disclosure and the claims, unless the context clearly indicates an exception, the words “a,” “an,” “one,” and/or “the” are not specifically singular and may also include plural. In general, the terms “include” and “contain” only indicate including explicitly identified steps and elements. These steps and elements do not constitute an exclusive list. A method or device may also include other steps or elements.

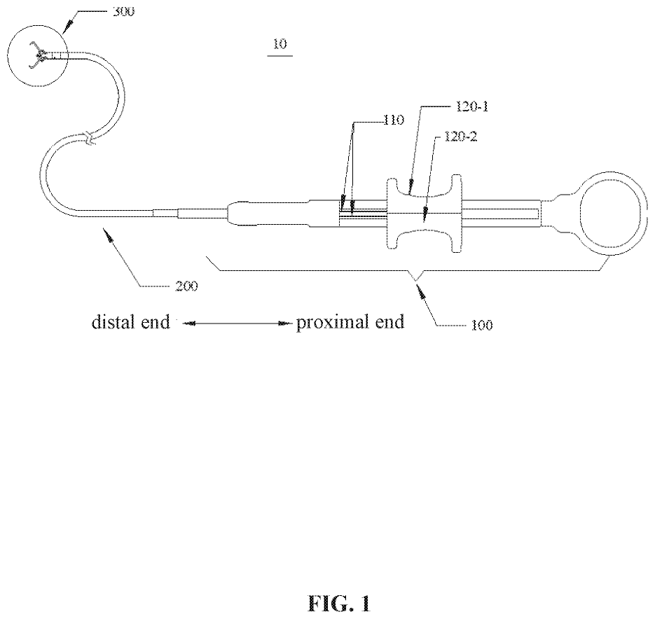

FIG. 1 is a schematic structural diagram of a clip instrument 10 according to some embodiments of the present disclosure.

As shown in FIG. 1, the clip instrument 10 may be applied to a medical endoscope to perform operations such as clamping, hemostasis, and suturing in endoscopic surgery. In some embodiments, the clip instrument 10 includes a control device 100, a delivery device 200, and a clip device 300. The clip device 300 is releasably connected with the delivery device 200. In some embodiments, the clip device 300 is releasably disposed at a distal end of the delivery device 200. The delivery device 200 is configured to deliver the clip device 300 into a human body through an endoscopic channel. After the control device 100 controls the clip device 300 to perform opening, closing, locking, and releasing operations, the clip device 300 remains in the human body. After the delivery device 200 is separated from the clip device 300, the delivery device 200 is withdrawn from the endoscopic channel.

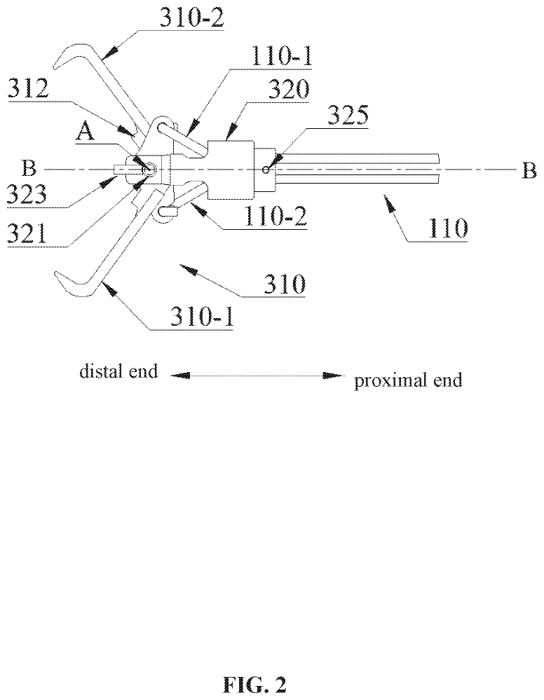

In some embodiments, the clip device 300 includes a clamping portion 310 (as shown in FIG. 2). To enable the clamping portion 310 to effectively clamp a wound 410 of tissue 400, a clamping direction of the clamping portion 310 needs to face the wound 410. In clinical surgery, an angle of a distal end of an endoscope body is adjusted to make the clamping portion 310 face the endoscope body. However, it is difficult to operate and inaccurate in positioning, leading to prolonged surgery time and increased risk.

In view of this, some embodiments of the present disclosure provide the clip instrument 10. The clamping portion 310 of the clip instrument 10 is configured to be capable of rotating in the same direction relative to a clip base 320. The clamping portion 310 may be adjusted to deflect relative to the distal end of the endoscope body to more conveniently make the clamping portion 310 face the tissue 400, thereby reducing operation difficulty. In some embodiments, the clip base 320 includes a limiting portion 323. The limiting portion 323 may limit a deflection angle of the clamping portion 310, which prevents the clamping portion 310 from deflecting too much in a direction D1 toward a central axis B-B of the clip base 320, prevents a clamping direction of the clamping portion 310 from misaligning with the wound 410, thereby preventing clamping failure and improving positioning accuracy of the clamping portion 310 facing the wound 410 during a deflection process of the clamping portion 310.

The terms “proximal end” and “distal end” involved in some embodiments of the present disclosure may represent an endpoint, an end face, an end portion, a portion near the end portion and having a certain length, etc., of the clip instrument 10 or a component thereof. Accordingly, “toward the proximal end” and “toward the distal end” refer to directions toward the corresponding endpoints. For example, an end of the clip instrument 10 inserted into a human body is the “distal end.” Accordingly, “toward the distal end” refers to a direction toward the end that is inserted into the human body, long an axis of the clip instrument 10. Another end (an endpoint, an end face, an end portion, a portion near the end portion and having a certain length, etc.) opposite to the distal end of the clip instrument 10 is the “proximal end.” Accordingly, “toward the proximal end” refers to a direction away from the end that is inserted into the human body, long the axis of the clip instrument 10.

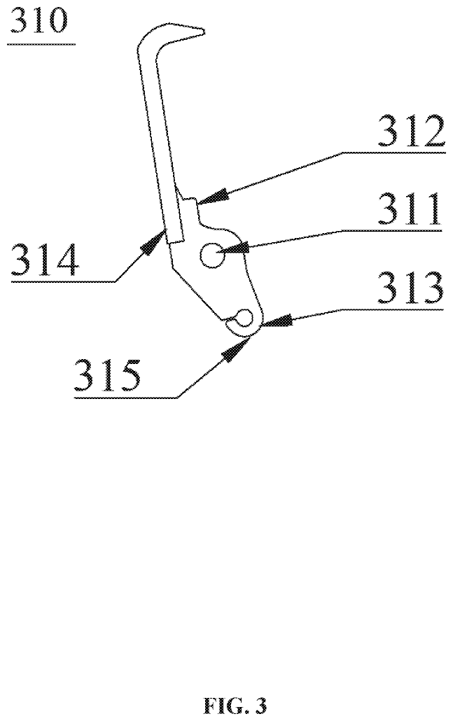

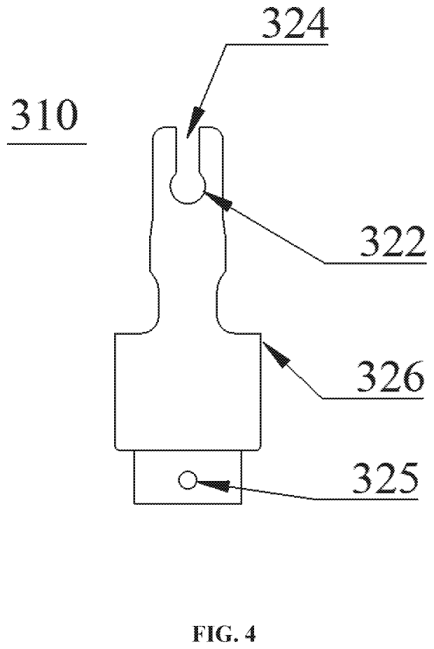

FIG. 2 is a schematic structural diagram of the clip device 300 according to some embodiments of the present disclosure. FIG. 3 is a schematic structural diagram of the clamping portion 310 according to some embodiments of the present disclosure. FIG. 4 is a schematic structural diagram of the clip base 320 according to some embodiments of the present disclosure.

As shown in FIG. 2, in some embodiments, the clip instrument 10 includes the clip device 300. The clip device 300 includes a clamping portion 310. The clamping portion 310 refers to a component configured to clamp the wound 410 of the tissue 400. In some embodiments, the clamping portion 310 may include a first clamping portion 310-1 and a second clamping portion 310-2. In some embodiments, the clamping portion 310 deflects about an axis A under an action of the control device 100. The axis A is perpendicular to a plane where the first clamping portion 310-1 and the second clamping portion 310-2 (hereinafter referred to as the two clamping portions) are located. For example, the axis A in FIG. 2 extends inward and outward relative to the plane of the drawing. In some embodiments, the control device 100 refers to a component configured to control deflection of the clamping portion 310. The control device 100 includes, but is not limited to, structures such as a control wire 110 and a control handle 120. In some embodiments, the control wire 110 may include a first control wire 110-1 and a second control wire 110-2.

In some embodiments, the clamping portions deflect about the axis A in a clockwise direction or a counterclockwise direction. For example, the two clamping portions may deflect about the axis A in opposite directions, i.e., one clamping portion (e.g., the first clamping portion 310-1) deflects about the axis A clockwise, and the other clamping portion (e.g., the second clamping portion 310-2) deflects about the axis A counterclockwise, which makes the clamping portion 310 perform opening or closing operations to achieve a clamping function. As another example, the two clamping portions may deflect about the axis A in the same direction, i.e., both the first clamping portion 310-1 and the second clamping portion 310-2 deflect about the axis A clockwise or counterclockwise, which makes the clamping portion 310 perform an integral deflection operation to achieve a function of positioning the clamping portion 310 to face the wound 410 of the tissue 400.

In clinical operations, after being opened, the first clamping portion 310-1 and the second clamping portion 310-2 are operated to deflect in the same direction to face the wound 410 of the tissue 400, which facilitates effective clamping of the wound 410 of the tissue 400 and ensures a clamping effect. Moreover, after operating the clamping portion 310 to open, an operator does not need to replace the control handle 120, then operates the clamping portion 310 to rotate through the control device 100, which is more convenient, simpler, and more accurate in positioning compared with replacing an endoscopic handle to operate the distal end of the endoscope to rotate, thereby saving surgical time and reducing the surgical risk.

As shown in FIG. 2 to FIG. 4, in some embodiments, the clip device 300 further includes the clip base 320. The clip base 320 refers to a structure configured to mount the clamping portion 310. The clamping portion 310 is rotatably fitted to the clip base 320. In some embodiments, one of the clamping portion 310 and the clip base 320 includes a pin, and the other includes a pin hole. The pin and the pin hole are rotatably fitted, so that the clamping portion 310 and the clip base 320 are rotatably connected. For example, the clamping portion 310 includes a pin hole 311. The clip base 320 includes a pin 321. The clamping portion 310 is fitted with the pin 321 of the clip base 320 through the pin hole 311, so that the clamping portion 310 and the clip base 320 are rotatably connected relative to each other. In some embodiments, the clip base 320 includes a mounting hole 322 and the pin 321. When connecting the clamping portion 310 and the clip base 320, the mounting hole 322 of the clip base 320 is aligned with the pin hole 311 of the clamping portion 310. The pin 321 is detachably inserted into the mounting hole 322 and the pin hole 311, which facilitates mounting the clamping portion 310 onto the clip base 320. In other embodiments, the clamping portion 310 is rotatably fitted to the clip base 320 through other structures.

In some embodiments, an axis of the pin 321 is perpendicular to the plane where the two clamping portions are located. The two clamping portions deflect about the axis of the pin 321.

In some embodiments, the two clamping portions may be respectively hinged to the clip base 320. A hinge point may be located between a proximal end and a distal end of each clamping portion. The hinge point refers to a position where the axis A, about which the clamping portion 310 deflects, is located. In some embodiments, the hinge points where the two clamping portions are respectively hinged to the clip base 320 are the same, which reduces assembly difficulty and enables the two clamping portions to be firmly connected.

In some embodiments, the first control wire 110-1 is connected to the proximal end of the first clamping portion 310-1. The second control wire 110-2 is connected to the proximal end of the second clamping portion 310-2. In some embodiments, the proximal end and the distal end of the first clamping portion 310-1 are respectively located on two sides of the central axis B-B of the clip base 320. The proximal end and the distal end of the second clamping portion 310-2 are respectively located on the two sides of the central axis B-B of the clip base 320. The proximal end of the first clamping portion 310-1 and the proximal end of the second clamping portion 310-2 are respectively located on the two sides of the central axis B-B of the clip base 320.

By setting the hinge point between the distal end and the proximal end of the clamping portion, a hook at the proximal end of the clamping portion can achieve a locking function. More description regarding the hook may be found in related description of FIG. 3-FIG. 6 below.

As shown in FIG. 2, in some embodiments, the clip base 320 includes the limiting portion 323. The limiting portion 323 is configured to limit a deflection angle of at least one of the two clamping portions (e.g., the first clamping portion 310-1 and the second clamping portion 310-2) relative to the clip base 320. For example, the limiting portion 323 is configured to limit the deflection angle of the first clamping portion 310-1 and/or the second clamping portion 310-2. In some embodiments, the limiting portion 323 contacts the clamping portion to form a stop during a process of the clamping portion deflecting in the direction D1 toward the central axis B-B of the clip base 320 and/or a direction D2 away from the central axis B-B of the clip base 320, which prevents the clamping portion from continuing to deflect, thereby limiting the deflection angle of the clamping portion. The clip base 320 limits the deflection angle of the clamping portion through the limiting portion 323. When the two clamping portions deflect in the same direction, the limiting portion 323 can prevent the clamping portion 310 from deflecting at an excessive angle beyond a target deflection angle range and avoid a situation where the clamping portion 310 cannot accurately face the wound 410 of the tissue 400. Moreover, the limiting portion 323 limits the clamping portion 310 to deflect within a specific angle range, which avoids accidents such as scratching the endoscopic channel or other unexpected accidents.

As shown in FIG. 2 to FIG. 4, in some embodiments, the limiting portion 323 is located between the two clamping portions. Each of the two clamping portions includes a stop portion 312. When the clamping portion deflects in the direction D1 toward the central axis B-B of the clip base 320 to a preset position, the corresponding stop portion 312 contacts the limiting portion 323 to stop. In some embodiments, the preset position includes a position on the central axis B-B of the clip base 320. When the clamping portion deflects in the direction D1 toward the central axis B-B of the clip base 320 to a position collinear with the central axis B-B of the clip base 320, the stop portion 312 contacts the limiting portion 323 to stop. In some embodiments, the preset position includes a position that forms a preset angle with the central axis B-B of the clip base 320, and the preset angle is within a range of 0 to 30 degrees. For example, the preset angle is 10 degrees. When the clamping portion deflects toward the direction D1 toward the central axis B-B of the clip base 320 to a position that forms a 10-degree angle with the central axis B-B of the clip base 320, the corresponding stop portion 312 contacts the limiting portion 323 and stops. By arranging the limiting portion 323 between the two clamping portions and by causing the limiting portion 323 to contact and stop the clamping portion when the clamping portion deflects inward, excessive deflection of the clamping portion toward the direction D1 toward the central axis B-B of the clip base 320 can be avoided, which prevents misalignment between the clamping direction of the two clamping portions and the wound 410, thereby preventing clamping failure and improving positioning accuracy of the clamping portion 310 in facing the wound 410 during the deflection process. Furthermore, the limiting portion 323 can also prevent an included angle between the two clamping portions from becoming too small, which would prevent effective clamping of the wound 410.

In some embodiments, the limiting portion 323 is disposed between the two clamping portions. When either one of the two clamping portions deflects to the position of the limiting portion 323, the limiting portion 323 restricts further deflection of the clamping portion 310. In some embodiments, the limiting portion 323 is arranged at a preset angle relative to the central axis B-B of the clip base 320, and only functions to restrict deflection for one of the two clamping portions. Merely by way of example, when deflects toward the limiting portion 323, one of the two clamping portions deflects to the position of the limiting portion 323, abuts against the limiting portion 323, and stops deflecting, while the other one of the two clamping portions stops deflecting before contacting the limiting portion 323 due to certain factors (e.g., constraint by the control wire or obstruction by tissue). Therefore, the limiting portion 323 only functions to limit one of the two clamping portions.

In some embodiments, the limiting portion 323 is disposed at a distal end of the clip base 320. In some embodiments, the distal end of the clip base 320 includes a mounting groove 324. The limiting portion 323 is configured as a sheet-like structure and is detachably fixed within the mounting groove 324. In some embodiments, the mounting groove 324 communicates with a mounting hole 322 of the clip base 320. The pin 321 connecting the clamping portion 310 and the clip base 320 may enter the mounting hole 322 from the mounting groove 324, thereby reducing assembly difficulty. In some embodiments, the limiting portion 323 may be a protrusion or other structures at the distal end of the clip base 320.

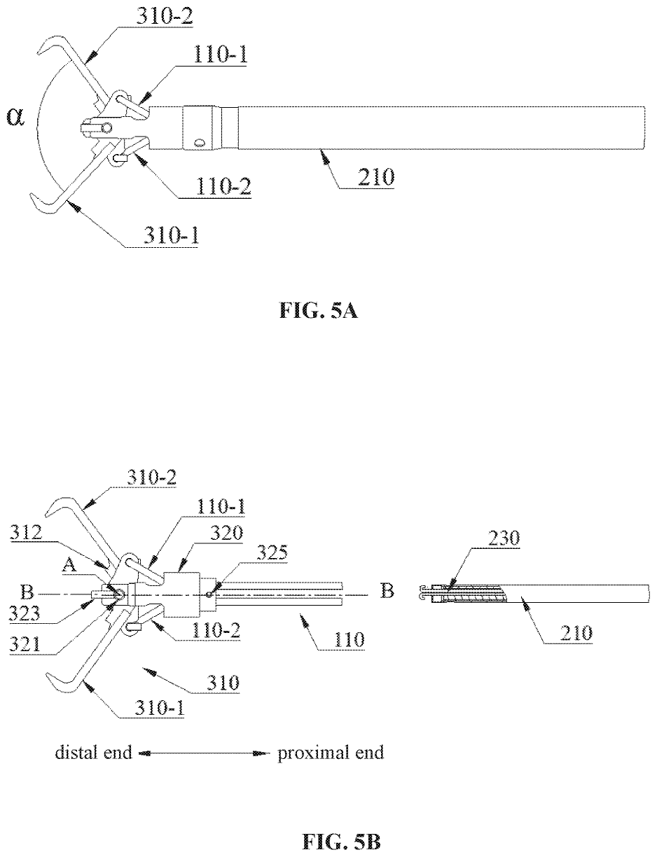

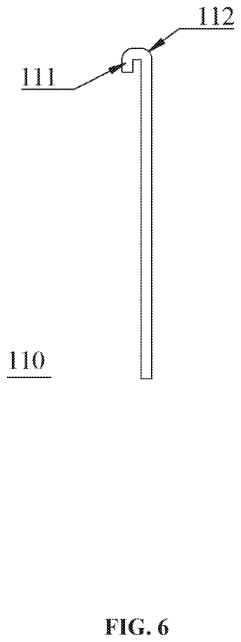

FIG. 5A is a schematic diagram of the clip device 300 and the delivery device 200 according to some embodiments of the present disclosure. FIG. 5B is an exploded schematic diagram of the clip device 300 and the delivery device 200 according to some embodiments of the present disclosure. FIG. 6 is a schematic structural diagram of the control wire 110 according to some embodiments of the present disclosure.

As shown in FIG. 5A and FIG. 5B, in some embodiments, the clip instrument 10 further includes the delivery device 200. The delivery device 200 includes a sheath 210 and the control wire 110 disposed within the sheath 210. The control wire 110 refers to a metal wire for manipulating the clamping portion 310. In some embodiments, the control wire 110 includes the first control wire 110-1 and the second control wire 110-2. The clamping portion 310 includes the first clamping portion 310-1 and the second clamping portion 310-2. The first control wire 110-1 is releasably connected to the first clamping portion 310-1. The second control wire 110-2 is releasably connected to the second clamping portion 310-2. Herein, “releasably connected” refers to a state where two components remain connected when a preset requirement is met (e.g., when an external force applied is less than a preset threshold) and separate from each other when the preset requirement is not met (e.g., when the external force applied is greater than the preset threshold). The first control wire 110-1 involved in the present disclosure refers to one of two control wires, and the second control wire 110-2 refers to the other one of the two control wires, and is not limited to the specific control wire indicated in the drawings. Similarly, the first clamping portion 310-1 refers to one of the two clamping portions, and the second clamping portion 310-2 refers to the other one of the two clamping portions, and is not limited to the specific clamping portion indicated in the drawings.

In some embodiments, the first control wire 110-1 moves from the distal end toward the proximal end of the first clamping portion 310-1 to control the first clamping portion 310-1 to deflect in the direction away from the central axis B-B of the clip base 320. The second control wire 110-2 moves from the distal end toward the proximal end of the second clamping portion 310-2 to control the second clamping portion 310-2 to deflect in the direction away the central axis B-B of the clip base 320. The clamping portion 310 is in an open state. In some embodiments, a maximum included angle α formed after the first clamping portion and the second clamping portion are opened is less than 170 degrees, so as to prevent the first clamping portion 310-1 and the second clamping portion 310-2 from opening too wide and damaging the endoscopic channel (or the working channel). For example, the maximum included angle α formed after deflection of the first clamping portion 310-1 and the second clamping portion 310-2 includes, but is not limited to, 120 degrees, 130 degrees, 145 degrees, 150 degrees, 160 degrees, etc.

As shown in FIG. 3 to FIG. 6, in some embodiments, the clamping portion 310 includes a hook 313 disposed at the proximal end of the clamping portion 310. The control wire 110 includes a curved hook 111 disposed at the distal end of the control wire 110. The hook 313 engages with the curved hook 111, and the clamping portion 310 is releasably connected to the control wire 110. In some embodiments, movement of the control wire 110 from the distal end toward the proximal end causes deformation, displacement, or breakage of the hook 313, thereby releasing the engagement between the curved hook 111 and the hook 313 and releasing the connection between the control wire 110 and the clamping portion 310.

In some embodiments, the clamping portion 310 includes a locked portion 315. The clip base 320 includes a locking portion 326. When the control wire 110 moves from the distal end toward the proximal end to close the two clamping portions, the locked portion 315 extends axially to engage with the locking portion 326, and the clip device 300 is locked. In some embodiments, the clamping portion 310 includes the hook 313 disposed at the proximal end of the clamping portion 310. The hook 313 constitutes the locked portion 315. The distal end of the clip base 320 constitutes the locking portion 326. When the control wire 110 moves from the distal end toward the proximal end to close the two clamping portions, a portion of the hook 313 deforms to increase its axial length, so that the hook 313 extends into the clip base 320 and engages with the clip base 320, and the clip device 300 is locked. Locking of the locked portion 315 and the locking portion 326 is controlled by an axial force, without additional forces or actions in other directions, which simplifies the structure and operation of the clip device 300 and reduces the probability of failure during the locking process.

In some embodiments, the control wire 110 is releasably connected to the hook 313. After the clip device 300 is locked, the connection between the control wire 110 and the hook 313 is released.

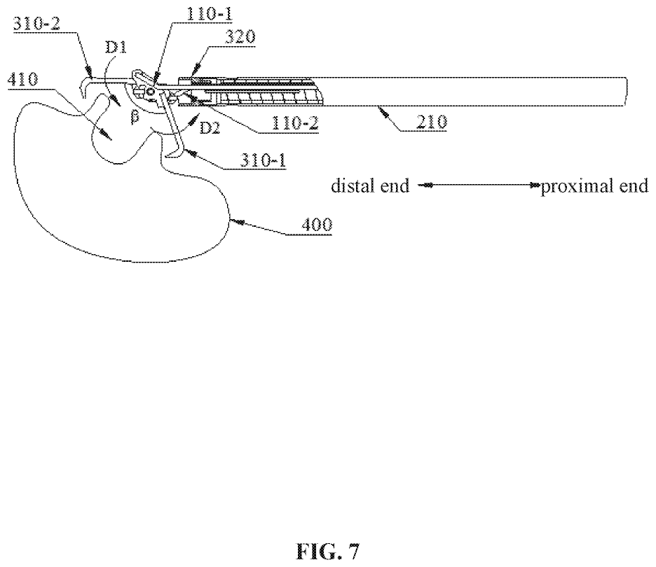

FIG. 7 is a schematic diagram of deflection of the clip instrument 10 according to some embodiments of the present disclosure.

As shown in FIG. 7, in some embodiments, the two clamping portions deflect in the same direction around the axis A under the action of the first control wire 110-1 and the second control wire 110-2. The axis A is perpendicular to a plane where the two clamping portions are located. In some embodiments, the first control wire 110-1 pushes from the proximal end toward the distal end to cause the first clamping portion 310-1 to deflect in the direction D2 away from the central axis B-B of the clip base 320. The second control wire 110-2 pulls from the distal end toward the proximal end to cause the second clamping portion 310-2 to deflect in the direction D1 toward the central axis B-B of the clip base 320. The two clamping portions (i.e., the first clamping portion 310-1 and the second clamping portion 310-2) deflect in the same direction around the axis A. Specifically, the distal end of the first control wire 110-1 is connected to the proximal end of the first clamping portion 310-1. When the first control wire 110-1 moves from the proximal end toward the distal end, the proximal end of the first clamping portion 310-1 is subjected to a pushing force, causing the first clamping portion 310-1 to pivot around the pin 321 and deflect in the direction D2 away from the central axis B-B of the clip base 320. The distal end of the second control wire 110-2 is connected to the proximal end of the second clamping portion 310-2. When the second control wire 110-2 moves from the distal end toward the proximal end, the proximal end of the second clamping portion 310-2 is subjected to a pulling force, causing the second clamping portion 310-2 to pivot around the pin 321 and deflect in the direction D1 toward the central axis B-B of the clip base 320. By using the first control wire 110-1 to control deflection of the first clamping portion 310-1 and using the second control wire 110-2 to control deflection of the second clamping portion 310-2, the operation is simple and fast. Furthermore, movement of the first clamping portion 310-1 and movement of the second clamping portion 310-2 do not interfere with each other. Therefore, the operator can adjust the first clamping portion 310-1 and the second clamping portion 310-2 to deflect sequentially or synchronously according to actual conditions, making the operation more flexible.

In some embodiments, the first control wire 110-1 may control a deflection angle of the first clamping portion 310-1. The second control wire 110-2 may control a deflection angle of the second clamping portion 310-2. In some embodiments, the deflection angle of the first clamping portion 310-1 and the deflection angle of the second clamping portion 310-2 are the same. In some embodiments, the deflection angle of the first clamping portion 310-1 and the deflection angle of the second clamping portion 310-2 are different. By separately adjusting the deflection angle of the first clamping portion 310-1 and the deflection angle of the second clamping portion 310-2, the operator can make a clamping space between the first clamping portion 310-1 and the second clamping portion 310-2 more closely match a size of the wound 410, thereby achieving more precise closure of the wound 410.

In some embodiments, a maximum included angle β formed after the first clamping portion 310-1 and the second clamping portion 310-2 deflect is less than 170 degrees, so as to prevent the first clamping portion 310-1 and the second clamping portion 310-2 from opening too wide and damaging the endoscopic channel or the sheath 210. For example, the maximum included angle β formed after the first clamping portion 310-1 and the second clamping portion 310-2 deflect includes, but is not limited to, 120 degrees, 130 degrees, 145 degrees, 150 degrees, 160 degrees, etc.

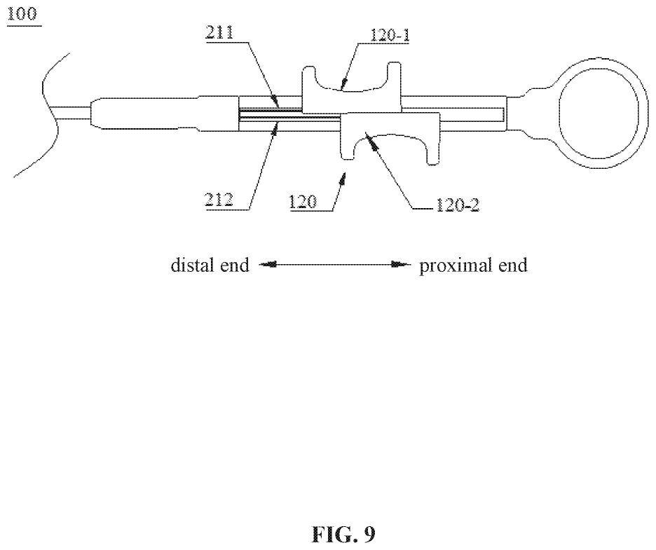

In some embodiments, the control device 100 includes a control handle 120. The control handle 120 is for the operator to manipulate the control wire 110. For example, by moving the control handle 120 to pull the control wire 110, the control wire 110 moves relative to the sheath 210 from the distal end toward the proximal end and/or from the proximal end toward the distal end. In some embodiments, the control device 100 includes two control handles. The first control wire 110-1 and the second control wire 110-2 are controlled by the two control handles, respectively. More description regarding the control handle 120 may be found in FIG. 9 and related description thereof.

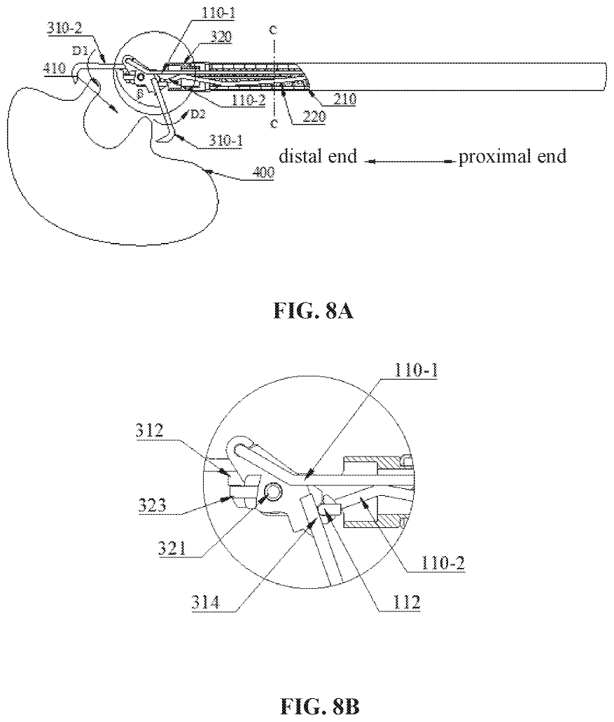

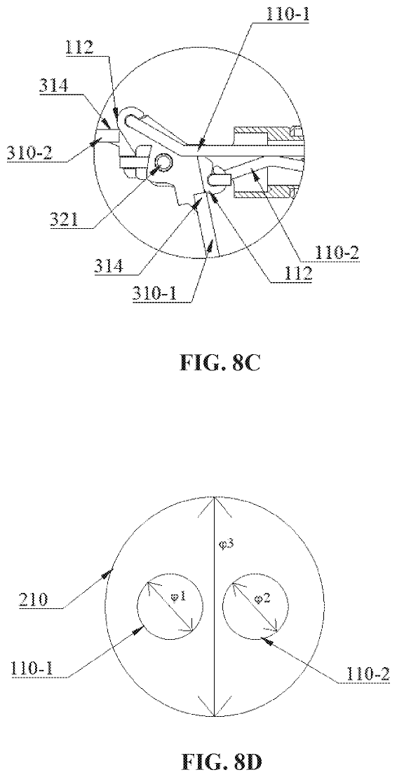

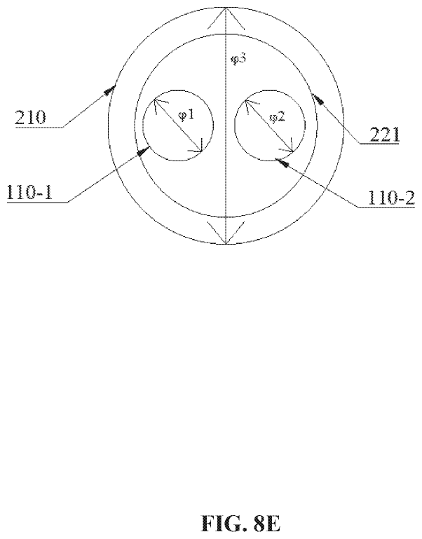

FIG. 8A is a schematic diagram of deflection of the clip instrument 10 according to some embodiments of the present disclosure.

As shown in FIG. 8A, in some embodiments, the two clamping portions deflect in the same direction around the axis A under the action of the first control wire 110-1 or the second control wire 110-2. The axis A is perpendicular to a plane where the two clamping portions is located. In some embodiments, when the distal end of the first clamping portion 310-1 is subjected to an external force, the first clamping portion 310-1 deflects in the direction D2 away from the central axis B-B of the clip base 320, which drives the second clamping portion 310-2 to deflect in the direction D1 toward the central axis B-B of the clip base 320, while simultaneously causing deformation of the second control wire 110-2. The two clamping portions (i.e., the first clamping portion 310-1 and the second clamping portion 310-2) deflect in the same direction around the axis A. The central axis B-B of the clip base 320 refers to a line passing through a center of a cross-section of the clip base 320 and extending in an axial direction. When the distal end of the first clamping portion 310-1 is subjected to the external force, the first clamping portion 310-1 deflects in the direction D2 away from the central axis B-B of the clip base 320, which directly or indirectly drive the second clamping portion 310-2 to deflect in the direction D1 toward the central axis B-B of the clip base 320. For example, after the first clamping portion 310-1 deflects, the first clamping portion 310-1 abuts against the second clamping portion 310-2, thereby directly driving the second clamping portion 310-2 to deflect. As another example, after the first clamping portion 310-1 deflects, the first clamping portion 310-1 abuts against the second control wire 110-2, and the second control wire 110-2 drives the second clamping portion 310-2 to deflect, thereby indirectly driving the second clamping portion 310-2 to deflect.

Specifically, when the distal end of the first clamping portion 310-1 abuts against an external object, by controlling the sheath 210 or the clamping portion 310 to move toward the distal end, the distal end of the first clamping portion 310-1 is subjected to pressure and deflects in the direction D2 away from the central axis B-B of the clip base 320. The external object includes, but is not limited to, the tissue 400 of the human body, a model of the tissue 400, or other structures. When deflects in the direction D2 away from the central axis B-B of the clip base 320, the first clamping portion 310-1 may cause the second control wire 110-2 to deform and drive the second clamping portion 310-2 to deflect in the direction D1 toward the central axis B-B of the clip base 320. The second control wire 110-2 deforms by abutting against the first clamping portion 310-1. By applying a force to the first clamping portion 310-1 through the external object to cause the first clamping portion 310-1 to deflect, and by driving the second clamping portion 310-2 to deflect through the deflection of the first clamping portion 310-1, the operator does not need to operate the first clamping portion 310-1 and the second clamping portion 310-2, and only needs to adjust the endoscope to achieve deflection of the two clamping portions, which simplifies the operation. Furthermore, the first clamping portion 310-1 may abut against an edge of the wound 410 and then deflect, which can more accurately orient the clamping space of the clamping portion 310 toward the wound 410, achieving precise positioning.

Similarly, in some embodiments, when the distal end of the second clamping portion 310-2 is subjected to an external force, the second clamping portion 310-2 deflects in the direction away from the central axis B-B of the clip base 320, and causes the first control wire 110-1 to deform and drive the first clamping portion 310-1 to deflect in the direction toward the central axis B-B of the clip base 320.

In some embodiments, the maximum included angle β formed after the first clamping portion 310-1 and the second clamping portion 310-2 deflect is less than 170 degrees, so as to prevent the first clamping portion 310-1 and the second clamping portion 310-2 from opening too wide and damaging the endoscopic channel or the sheath 210. For example, the maximum included angle β formed after the first clamping portion 310-1 and the second clamping portion 310-2 deflect includes, but is not limited to, 120 degrees, 130 degrees, 145 degrees, 150 degrees, 160 degrees, etc.

FIG. 8B is a partial enlarged schematic diagram of the clip instrument 10 according to some embodiments shown in FIG. 8A.

As shown in FIG. 3, FIG. 6, and FIG. 8B, in some embodiments, the clamping portion includes a contact portion 314. In some embodiments, the contact portion 314 includes, but is not limited to, a protrusion or a recess provided on an outer side of the clamping portion, or a local structure of the clamping portion. The outer side of the clamping portion 310 refers to a side indicated by an arrow in the drawings.

In some embodiments, the distal end of each of the control wires includes a mating portion 112. In some embodiments, the mating portion 112 includes, but is not limited to, a recess or a protrusion provided at the distal end of the control wire, or a local structure of the control wire.

In some embodiments, when the first clamping portion 310-1 deflects in the direction D2 away from the central axis B-B of the clip base 320, the contact portion 314 of the first clamping portion 310-1 abuts against the mating portion 112 of the second control wire 110-2, causing the second control wire 110-2 to deform. Specifically, the contact portion 314 of the first clamping portion 310-1 abuts against the mating portion 112 of the second control wire 110-2. The first clamping portion 310-1 continues to deflect in the direction D2 away from the central axis B-B of the clip base 320, causing at least a portion of the distal end of the second control wire 110-2 to be compressed and deform. After the second control wire 110-2 deforms, the second control wire 110-2 drives the second clamping portion 310-2 to deflect in the direction D1 toward the central axis B-B of the clip base 320.

In some embodiments, after the first clamping portion 310-1 abuts against the second control wire 110-2 or the second clamping portion 310-2, an included angle between the first clamping portion 310-1 and the second clamping portion 310-2 remains unchanged. In this way, the first clamping portion 310-1 and the second clamping portion 310-2 maintain the unchanged included angle and deflect integrally, aligning the clamping space between the first clamping portion 310-1 and the second clamping portion 310-2 with the wound 410.

In some embodiments, a force for the deformation of the second control wire 110-2 is in a range of 0.5 to 2 N. In some embodiments, the force for the deformation of the second control wire 110-2 is in a range of 0.8 to 1.5 N. In some embodiments, the force for the deformation of the second control wire 110-2 is, but not limited to, 0.5 N, 0.8 N, 1.0 N, 1.2 N, 1.5 N, 2 N, etc.

FIG. 8C is a partial enlarged schematic diagram of the clip instrument 10 according to other embodiments of FIG. 8A.

As shown in FIG. 8C, in some embodiments, each clamping portion includes the contact portion 314 and the mating portion 112. In some embodiments, the contact portion 314 is located at a shoulder of the clamping portion, and the mating portion 112 is located at the hook 313 of the clamping portion.

In some embodiments, when the first clamping portion 310-1 deflects in the direction D2 away from the central axis B-B of the clip base 320, the contact portion 314 of the first clamping portion 310-1 abuts against the mating portion 112 of the second clamping portion 310-2, and/or the contact portion 314 of the second clamping portion 310-2 abuts against the mating portion 112 of the first clamping portion 310-1, which make the first clamping portion 310-1 push the second clamping portion 310-2 to deflect in the direction D2 away from the central axis B-B of the clip base 320, and make the second clamping portion 310-2 push the second control wire 110-2 to be compressed and deform, thereby achieving the integral deflection of the clamping portion 310.

In some embodiments, similar to the related description of FIG. 8B, after the first clamping portion 310-1 abuts against the second control wire 110-2 or the second clamping portion 310-2, the included angle between the first clamping portion 310-1 and the second clamping portion 310-2 remains unchanged.

FIG. 8D is a first simplified cross-sectional diagram of the clip instrument 10 taken along a line C-C according to some embodiments of FIG. 8A. FIG. 8E is a second simplified cross-sectional diagram of the clip instrument 10 taken along the line C-C according to some embodiments of FIG. 8A.

As shown in FIG. 8A to FIG. 8D, in some embodiments, the sheath 210 includes an accommodation space 220 for accommodating the deformed second control wire 110-2. In some embodiments, the sheath 210 has a hollow channel 213. At least a portion of the hollow channel 213 constitutes the accommodation space 220. The second control wire 110-2 is unconstrained within the accommodation space 220, and therefore may deform when subjected to a force. After deforms, the second control wire 110-2 may be bent and accommodated within the accommodation space 220. In some embodiments, the sheath 210 includes a lubrication tube 221. The first control wire 110-1 and the second control wire 110-2 are accommodated within the lubrication tube 221. A hollow channel of the lubrication tube 221 constitutes the accommodation space 220. The lubrication tube 221 can reduce friction experienced by the control wire 110 during movement.

In some embodiments, a radial dimension of the first control wire 110-1 is φ1, a radial dimension of the second control wire 110-2 is φ2, and a radial dimension of the accommodation space 220 is φ3. A ratio of a sum of the radial dimensions of the first control wire 110-1 and the second control wire 110-2 to the radial dimension of the accommodation space 220 is expressed by (φ1+φ2)/φ3, which is equal to 0.5. In some embodiments, an interior of the sheath 210 constitutes the accommodation space 220, and the radial dimension of the accommodation space 220 equals an inner diameter of the sheath 210. In other embodiments, an interior of the lubrication tube 221 constitutes the accommodation space 220, and the radial dimension of the accommodation space 220 equals an inner diameter of the lubrication tube 221.

In some embodiments, the radial dimension φ1 of the first control wire 110-1 is not less than 0.2 mm and not greater than 1.0 mm, the radial dimension φ2 of the second control wire 110-2 is not less than 0.2 mm and not greater than 1.0 mm, and the radial dimension φ3 of the accommodation space 220 is not less than 0.4 mm and not greater than 2.0 mm. In some embodiments, the radial dimension φ1 of the first control wire 110-1 is 0.3 mm, the radial dimension φ2 of the second control wire 110-2 is 0.3 mm, and the radial dimension φ3 of the accommodation space 220 is 1.2 mm. Under these dimensional conditions, the first control wire 110-1 or the second control wire 110-2 has sufficient space to deform, and the deformed and bent first control wire 110-1 or the deformed and bent second control wire 110-2 may be accommodated within the accommodation space 220.

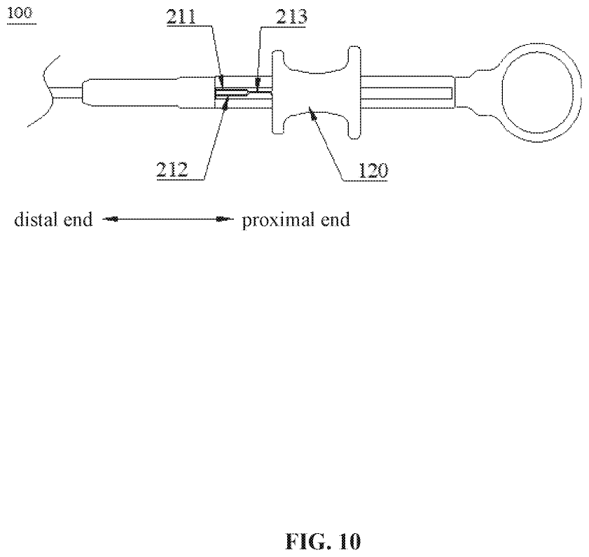

In some embodiments, the control device 100 includes one control handle 120. The first control wire 110-1 and the second control wire 110-2 are controlled simultaneously by the one control handle 120. In some embodiments, the control device 100 includes two control handles 120. The first control wire 110-1 and the second control wire 110-2 are controlled respectively by the two control handles 120. More embodiments regarding the control handle 120 may be found in FIG. 9 and FIG. 10 and related description thereof.

FIG. 9 is a schematic diagram of the control device 100 according to some embodiments of the present disclosure.

As shown in FIG. 7 to FIG. 9, in some embodiments, the control device 100 includes a first control handle 120-1 and a second control handle 120-2. The first control handle 120-1 is connected to a proximal end of the first control wire 110-1. The second control handle 120-2 is connected to a proximal end of the second control wire 110-2. During the clinical operation, the operator independently controls the two control wires through the first control handle 120-1 and the second control handle 120-2, respectively. This allows the movements of the two control wires to be independent of each other. The operator then uses the two control wires to independently actuate the two clamping portions, enabling the deflections of the clamping portions to be non-interfering. Consequently, operations such as opening, integral deflection, locking, closing, and releasing can be performed, offering greater operational flexibility.

In some embodiments, a first catheter 211 and a second catheter 212 that are independent of each other are disposed within the sheath 210. The first catheter 211 extends from the first control handle 120-1 to a distal end of the sheath 210 (e.g., to a proximal end of the accommodation space 220). The second catheter 212 extends from the second control handle 120-2 to the distal end of the sheath 210 (e.g., to the proximal end of the accommodation space 220). The first control wire 110-1 is movably disposed within the first catheter 211. The second control wire 110-2 is movably disposed within the second catheter 212. The first control handle 120-1 controls the movement of the first control wire 110-1 within the first catheter 211 by moving. The second control handle 120-2 controls the movement of the second control wire 110-2 within the second catheter 212 by moving. By providing the first catheter 211 and the second catheter 212 to separate the first control wire 110-1 and the second control wire 110-2, avoiding entanglement of the first control wire 110-1 and the second control wire 110-2 during the movement, thereby ensuring a smooth surgery.

In some embodiments, the first control handle 120-1 controls the first clamping portion 310-1 to deflect about the axis A through the first control wire 110-1. For example, the first control handle 120-1 moves from the distal end toward the proximal end, pulling the first control wire 110-1 to move from the distal end toward the proximal end, thereby causing the first clamping portion 310-1 to deflect in the direction toward the central axis B-B of the clip base 320. As another example, the first control handle 120-1 moves from the proximal end toward the distal end, pulling the first control wire 110-1 to move from the proximal end toward the distal end, thereby causing the first clamping portion 310-1 to deflect in the direction away from the central axis B-B of the clip base 320.

In some embodiments, the second control handle 120-2 controls the second clamping portion 310-2 to deflect about the axis A through the second control wire 110-2. For example, the second control handle 120-2 moves from the distal end toward the proximal end, pulling the second control wire 110-2 to move from the distal end toward the proximal end, thereby causing the second clamping portion 310-2 to deflect in the direction toward the central axis B-B of the clip base 320. As another example, the second control handle 120-2 moves from the proximal end to the distal end, pulling the second control wire 110-2 to move from the proximal end to the distal end, thereby causing the second clamping portion 310-2 to deflect in the direction away from the central axis B-B of the clip base 320.

FIG. 10 is a schematic diagram of the control device 100 according to some embodiments of the present disclosure.

As shown in FIG. 10, in some embodiments, the delivery device 200 includes one control handle 120. The control handle 120 is connected to the first control wire 110-1 and the second control wire 110-2. In clinical operations, when the operator moves the control handle 120, the first control wire 110-1 and the second control wire 110-2 move synchronously, so that the first clamping portion 310-1 and the second clamping portion 310-2 also deflect synchronously, which simplifies the operation. When the first clamping portion 310-1 and the second clamping portion 310-2 are operated through the control handle 120, the clamping portion 310 may perform operations such as opening, locking, closing, or releasing. The same-direction deflection of the two clamping portions may be achieved when the distal ends of the clamping portions contact and are forced by the tissue 400.

In some embodiments, the first catheter 211 and the second catheter 212 are provided inside the sheath 210. Proximal portions of the first catheter 211 and the second catheter 212 are merged into the same channel 213. The proximal portion of the first control wire 110-1 and the proximal portion of the second control wire 110-2 are merged together and accommodated in the channel 213. A remaining portion of the first control wire 110-1 is movably arranged in the first catheter 211, and a remaining portion of the second control wire 110-2 is movably arranged in the second catheter 212, which can avoid entanglement during the control process.

In some embodiments, the control handle 120 controls the first clamping portion 310-1 to deflect around the axis A by simultaneously controlling the first control wire 110-1 and the second control wire 110-2. For example, the control handle 120 moves from the proximal end to the distal end, drives the first control wire 110-1 and the second control wire 110-2 to simultaneously move from the proximal end to the distal end, so that the first clamping portion 310-1 and the second clamping portion 310-2 simultaneously deflect in the direction away from the central axis B-B of the clip base 320, and the two clamping portions open. As another example, the control handle 120 moves from the distal end to the proximal end, drives the first control wire 110-1 and the second control wire 110-2 to simultaneously move from the distal end to the proximal end, so that the first clamping portion 310-1 and the second clamping portion 310-2 simultaneously deflect in the direction toward the central axis B-B of the clip base 320, and the two clamping portions close.

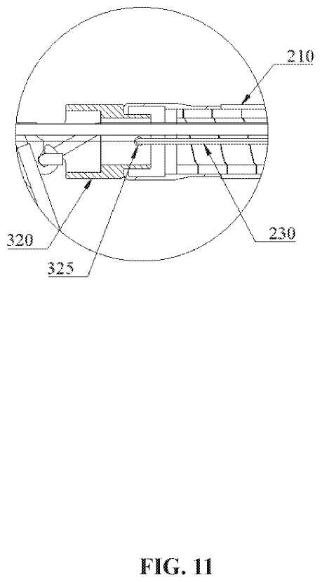

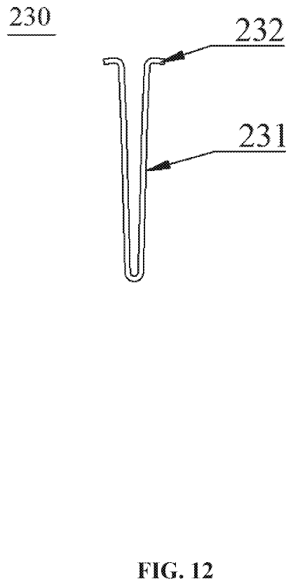

FIG. 11 is a partially enlarged schematic diagram of the clip instrument 10 according to some embodiments of the present disclosure. FIG. 12 is a schematic structural diagram of a connection pin 230 according to some embodiments of the present disclosure.

As shown in FIG. 4, FIG. 11, and FIG. 12, in some embodiments, the clip base 320 is rotatable around an axis of the sheath 210. The axis of the sheath 210 refers to a central axis in an extending direction of the sheath 210. The clip base 320 is rotatably connected to the sheath 210. By adjusting a rotation angle of the clip base 320, a closed direction of the two clamping portions can be made consistent with a closed direction of the wound 410 of the tissue 400, thereby effectively achieving closure of the wound 410 of the tissue 400.

In some embodiments, the delivery device 200 further includes the connection pin 230. The distal end of the sheath 210 is provided with a connection recess (not shown in the figure). The clip base 320 is provided with a connection hole 325. A distal end of the connection pin 230 passes through the connection hole 325 and extends into the connection recess, thereby connecting the clip base 320 to the sheath 210 and limiting an axial displacement between the clip base 320 and the sheath 210. In some embodiments, the connection recess may be an annular groove on an inner wall of the sheath 210. After the distal end of the connection pin 230 extends into the connection recess, the connection pin 230 can slide relative to the annular groove, thereby enabling the clip base 320 to rotate around the axis of the sheath 210. In other embodiments, the connection recess may also be a through hole or a point-shaped groove on a side wall of the sheath 210.

In some embodiments, the connection pin 230 includes at least two arms 231. Proximal ends of the arms 231 of the connection pin 230 are connected as a whole. Distal ends of the arms 231 are independent from each other and are formed with bent portions 232. The bent portions 232 are configured to pass through the connection hole 325 of the clip base 320 and the connection recess of the sheath 210 to connect the clip base 320 and the sheath 210.

In some embodiments, after the control wire 110 is disconnected from the clamping portion 310, the control wire 110 continues to move from the distal end to the proximal end. During the movement, the hook 111 of the control wire 110 hooks a proximal end of the connection pin 230, thereby pulling the connection pin 230 to disengage from the connection recess and the connection hole 325, and separating the clip base 320 from the sheath 210. In some embodiments, the distal end of the connection pin 230 disengages from the connection recess or the connection hole 325 through deformation, displacement, or fracture, thereby separating the clip base 320 from the sheath 210.

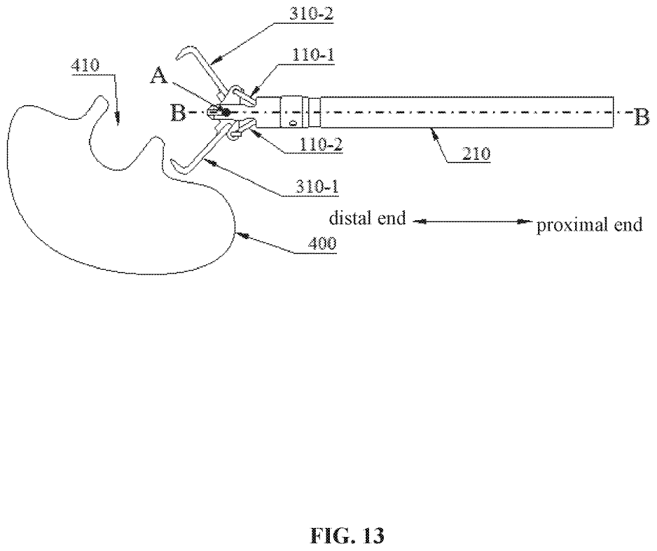

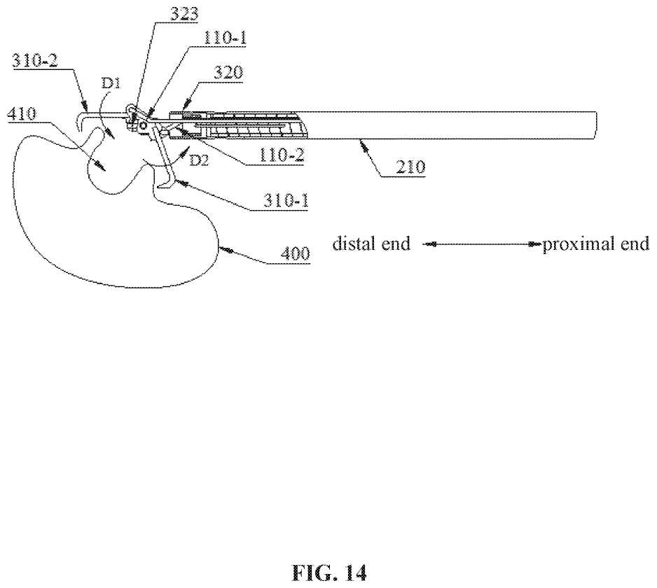

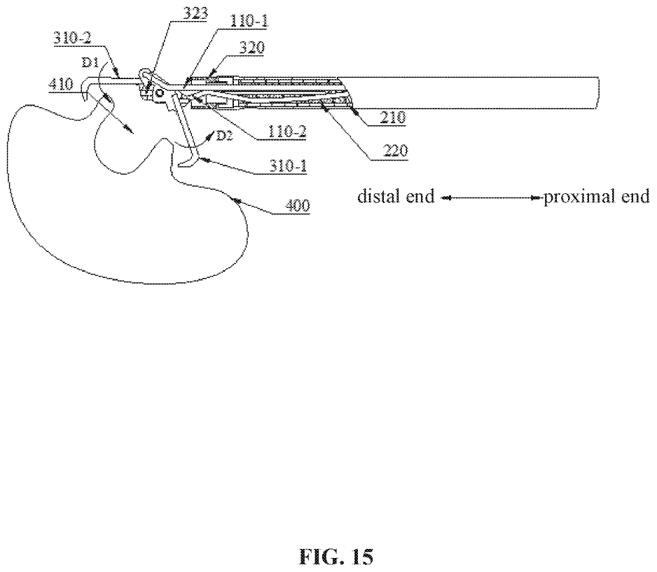

FIG. 13 is a schematic diagram of an open state of the clip instrument 10 according to some embodiments of the present disclosure. FIG. 14 is a schematic diagram of a deflected state of the clip instrument 10 according to some embodiments of the present disclosure. FIG. 15 is a schematic diagram of a deflected state of the clip instrument 10 according to other embodiments of the present disclosure.

In some embodiments, the clip instrument 10 includes an open state. The open state refers to a state in which the distal ends of the two clamping portions of the clip device 300 are separated. The open state may include a straight-open state and a side-open state. In the straight-open state, the two clamping portions are not deflected. The two clamping portions may be axisymmetric with the central axis B-B of the clip base as a symmetry axis, as shown in FIG. 13. In the side-open state, the two clamping portions deflect in the same direction around the axis A, as shown in FIG. 14 and FIG. 15. More description regarding the two clamping portions deflecting in the same direction around the axis A may be found in the related description of FIG. 8A above.

In some embodiments, because the movements of the two control handles are relatively independent, the movements of the corresponding two control wires are also independent of each other, and thus the movements of the two clamping portions are also independent of each other. When the control handle moves toward the distal end, the control handle drives the corresponding control wire to move toward the distal end, and the corresponding clamping portion deflects around the axis A in the direction away from the central axis B-B of the clip base. When the control handle moves toward the proximal end, the control handle drives the corresponding control wire to move toward the proximal end, and the corresponding clamping portion deflects around the axis A in the direction toward the central axis B-B of the clip base. When forces of different magnitudes and different directions are applied to the control handles, the clamping portion 310 can rotate around the axis A, thereby enabling the clip instrument 10 to transition among the straight-open state, the side-open state, and a closed state.

In some embodiments, when the first control wire 110-1 and/or the second control wire 110-2 act on the two clamping portions in a first preset mode, the clip instrument 10 switches from the straight-open state to the side-open state. The first preset mode at least includes: one of the first control wire 110-1 and the second control wire 110-2 moving toward the distal end. For example, the first control wire 110-1 moves toward the distal end, and drives the first clamping portion 310-1 to deflect in the direction D2 away from the central axis B-B of the clip base 320. The case where the second control wire 110-2 moves toward the distal end is similar and will not be repeated.

In some embodiments, the first preset mode includes: one of the first control wire 110-1 and the second control wire 110-2 moving toward the distal end, and the other of the first control wire 110-1 and the second control wire 110-2 moving toward the proximal end, thereby driving the first clamping portion 310-1 and the second clamping portion 310-2 to deflect in the same direction around the axis A. For example, the first control wire 110-1 moves toward the distal end and drives the first clamping portion 310-1 to deflect in the direction D2 away from the central axis B-B of the clip base 320, and simultaneously, the second control wire 110-2 moves toward the proximal end and drives the second clamping portion 310-2 to deflect in the direction D1 toward the central axis B-B of the clip base 320, thereby driving the first clamping portion 310-1 and the second clamping portion 310-2 to deflect in the same direction around the axis A, and switching the clip instrument 10 to the side-open state.

In some embodiments, when the first control wire 110-1 and/or the second control wire 110-2 act on the two clamping portions in a second preset mode, the clip instrument 10 switches from the side-open state to the straight-open state. The second preset mode includes: the other of the first control wire 110-1 and the second control wire 110-2 moving toward the distal end. For example, the first control wire 110-1 moves toward the distal end to control the clip instrument 10 to switch from the straight-open state to the side-open state. The second control wire 110-2 moves toward the distal end to switch the clip instrument 10 from the side-open state to the straight-open state. The second control wire 110-2 moves toward the distal end, and drives the second clamping portion 310-2 to deflect in a direction away from the central axis B-B of the clip base 320, which is opposite to the direction D1.

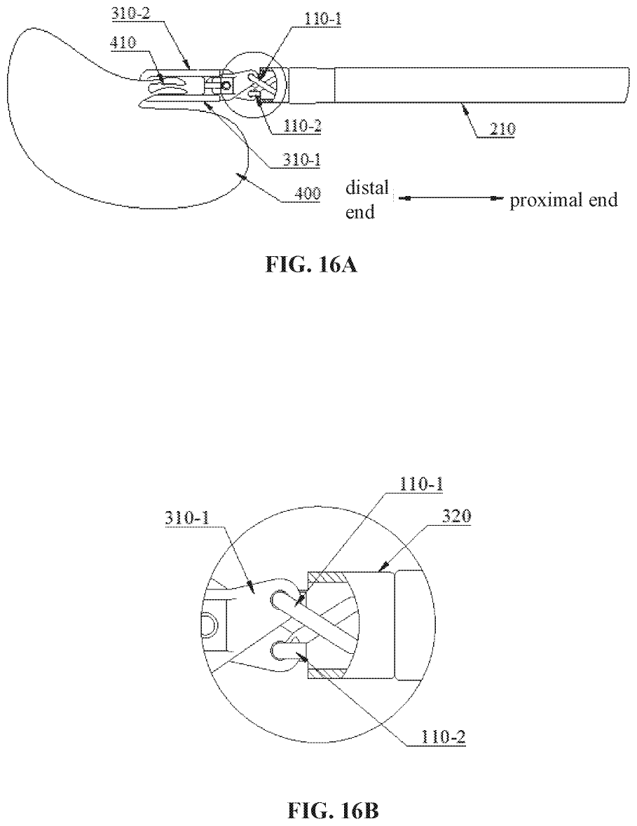

FIG. 16A is a schematic diagram of a closed state of the clip instrument 10 according to some embodiments of the present disclosure.

In some embodiments, as shown in FIG. 16A, the clip instrument 10 further includes a closed state. The closed state refers to a state in which the distal ends of the two clamping portions of the clip device 300 are in contact, and may also be referred as a clamping-closed state. In some embodiments, when the first control wire 110-1 and/or the second control wire 110-2 act on the two clamping portions in a third preset mode, the clip instrument 10 switches from the side-open state to the closed state. The third preset mode includes: among the distal ends of the first control wire 110-1 and the second control wire 110-2, the one that is more distal moving toward the proximal end. For example, as shown in FIG. 14, the distal end of the first control wire 110-1 is more distal than the distal end of the second control wire 110-2. The first control wire 110-1 moves toward the proximal end, and drives the first clamping portion 310-1 to deflect in the direction toward the central axis B-B of the clip base 320, which is opposite to the direction D2, so that the distal end of the first clamping portion 310-1 may move to contact the second clamping portion 310-2, causing the clip instrument 10 to be in the closed state.

In some embodiments, the third preset mode further includes: among the distal ends of the first control wire 110-1 and the second control wire 110-2, the one that is more proximal also moving toward the proximal end. For example, as shown in FIG. 14, the distal end of the second control wire 110-2 is more proximal than the distal end of the first control wire 110-1. The second control wire 110-2 moves toward the proximal end, driving the second clamping portion 310-2 to deflect in the direction D1 toward the central axis B-B of the clip base 320. Consequently, the first clamping portion 310-1 and the second clamping portion 310-2 move relative to each other until the distal ends of the first clamping portion 310-1 and the second clamping portion 310-2 contact, causing the clip instrument 10 to be in the closed state.

In some embodiments, as shown in FIG. 16A, the clip instrument 10 further includes the closed state. In some embodiments, when the first control wire 110-1 and the second control wire 110-2 act on the two clamping portions in a fourth preset mode, the clip instrument 10 switches from the straight-open state to the closed state. The fourth preset mode includes: the first control wire 110-1 and the second control wire 110-2 moving toward the proximal end. For example, the first control wire 110-1 moves toward the proximal end, driving the first clamping portion 310-1 to deflect in the direction toward the central axis B-B of the clip base 320, which is opposite to the direction D2. The second control wire 110-2 moves toward the proximal end, driving the second clamping portion 310-2 to deflect in the direction D1 toward the central axis B-B of the clip base 320. Consequently, the first clamping portion 310-1 and the second clamping portion 310-2 move relative to each other until the distal ends of the first clamping portion 310-1 and the second clamping portion 310-2 contact, causing the clip instrument 10 to be in the closed state.

In some embodiments, as shown in FIG. 16A, the clip instrument 10 further includes the closed state. In some embodiments, when the first control wire 110-1 and/or the second control wire 110-2 acts on the two clamping portions in a fifth preset mode, the clip instrument 10 switches from the closed state to the side-open state. The fifth preset mode includes: one of the first control wire 110-1 and the second control wire 110-2 moving toward the distal end. For example, the clip instrument 10 is in the closed state, and the distal ends of the two clamping portions contact, subsequently, the first control wire 110-1 moves toward the distal end, driving the first clamping portion 310-1 to deflect in the direction D2 away from the central axis B-B of the clip base 320. Consequently, the distal end of the first clamping portion 310-1 separates from the distal end of the second clamping portion 310-2, causing the clip instrument 10 to switch from the closed state to the side-open state.

In some embodiments, the fifth preset mode further includes: one of the first control wire 110-1 and the second control wire 110-2 moving toward the distal end by a first moving distance, and the other of the first control wire 110-1 and the second control wire 110-2 moving toward the distal end by a second moving distance, where the first moving distance is greater than the second moving distance. For example, the clip instrument 10 is in the closed state, and the distal ends of the two clamping portions contact. Subsequently, the first control wire 110-1 moves toward the distal end by the first moving distance, driving the first clamping portion 310-1 to deflect in the direction D2 away from the central axis B-B of the clip base 320. The second control wire 110-2 moves toward the distal end by the second moving distance, driving the second clamping portion 310-2 to deflect in the direction away from the central axis B-B of the clip base 320, which is opposite to the direction D1. Because the first moving distance is greater than the second moving distance, the deflection angle of the first clamping portion 310-1 may be greater than the deflection angle of the second clamping portion 310-2. Therefore, the distal end of the first clamping portion 310-1 separates from the distal end of the second clamping portion 310-2, and the clip instrument 10 switches from the closed state to the side-open state.

More description regarding how the control wire 110 drives the clamping portion 310 to achieve deflection may be found in the related description of FIG. 7 to FIG. 8C.

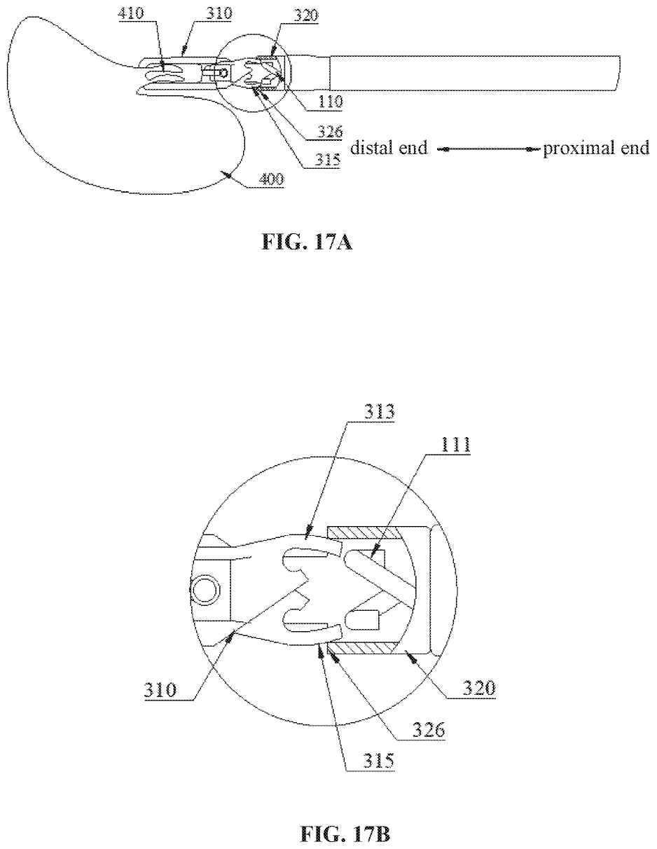

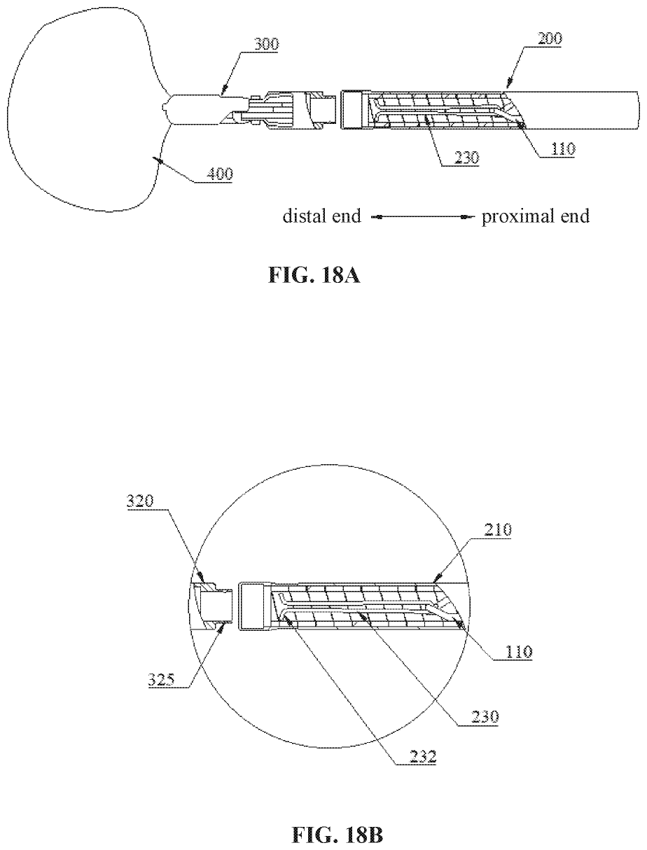

FIG. 16B is a partially enlarged schematic diagram of the clip instrument 10 according to some embodiments of FIG. 16A. FIG. 17A is a schematic diagram of a locked state of the clip instrument 10 and released states of the clamping portion and the control wire according to some embodiments of the present disclosure. FIG. 17B is a partially enlarged schematic diagram of the clip instrument 10 according to some embodiments of FIG. 17A. FIG. 18A is a schematic diagram of a released state of the clip device 300 and the delivery device 200 according to some embodiments of the present disclosure. FIG. 18B is a partially enlarged schematic diagram of the clip instrument 10 according to some embodiments of FIG. 18A. FIG. 13 to FIG. 18B are schematic diagrams of a motion process of the clip instrument 10 according to some embodiments of the present disclosure. The following describes operations, such as opening, deflecting integrally, closing, locking, and releasing, performed by the clip instrument 10 with reference to FIG. 13 to FIG. 18B. However, the following operations are merely illustrative, and the working process is not limited to the following operations.

In operation I, as shown in FIG. 13, after the clamping portion 310 is delivered to a target position (e.g., near the wound 410 of the tissue 400) through the delivery device 200, the first clamping portion 310-1 and the second clamping portion 310-2 open. In some embodiments, the first control wire 110-1 is connected to a first hook 313 of the first clamping portion 310-1. The first control wire 110-1 moves from the proximal end to the distal end to drive the first clamping portion 310-1 to deflect around the axis A in the direction away from the central axis B-B of the clip base 320. Correspondingly, the second control wire 110-2 is connected to a second hook 313 of the second clamping portion 310-2. The second control wire 110-2 moves from the proximal end to the distal end, driving the second clamping portion 310-2 to deflect around the axis A in the direction away from the central axis B-B of the clip base 320. At this time, the first clamping portion 310-1 and the second clamping portion 310-2 move away from each other and are in an open state. The clamping space is formed between the first clamping portion 310-1 and the second clamping portion 310-2, and the clamping space may clamp the wound 410 of the tissue 400.

In operation II, after the clamping portion 310 opens, a relative position between the wound 410 of the tissue 400 and the clamping space is determined, and the clamping portion 310 is controlled to deflect integrally to face the wound 410 according to the relative position.

As shown in FIG. 14, in some embodiments, the control device 100 includes the two control handles. When the first control handle 120-1 controls the first control wire 110-1 to move from the proximal end to the distal end, the proximal end of the first clamping portion 310-1 receives a pushing force, causing the first clamping portion 310-1 to deflect around the pin 321 in the direction D2 away from the central axis B-B of the clip base 320. The distal end of the second control wire 110-2 is connected to the proximal end of the second clamping portion 310-2. When the second control wire 110-2 moves from the distal end to the proximal end, the proximal end of the second clamping portion 310-2 receives a pulling force, causing the second clamping portion 310-2 to deflect around the pin 321 in the direction D1 toward the central axis B-B of the clip base 320. A clamping direction of the integral deflected clamping portion 310 may face the wound 410, achieving effective clamping.

As shown in FIG. 15, in other embodiments, the position of the clip device 300 is adjusted so that one of the two clamping portions of the clip device 300 abuts against an edge of the wound 410 of the tissue 400. When the distal end of the first clamping portion 310-1 abuts against the tissue 400, by controlling the sheath 210 or the clamping portion 310 to move toward the distal end, the distal end of the first clamping portion 310-1 receives a pressure and deflects in the direction D2 away from the central axis B-B of the clip base 320. When the first clamping portion 310-1 deflects in the direction D2 away from the central axis B-B of the clip base 320, the contact portion 314 of the first clamping portion 310-1 and the mating portion 112 of the second control wire 110-2 form an abutment. The first clamping portion 310-1 continues to deflect in the direction D2 away from the central axis B-B of the clip base 320, causing at least a portion of the distal end of the second control wire 110-2 to be squeezed and deform. After the second control wire 110-2 deforms, the second control wire 110-2 drives the second clamping portion 310-2 to deflect in the direction D1 toward the central axis B-B of the clip base 320. The clamping direction of the integral deflected clamping portion 310 may face the wound 410, achieving effective clamping.

In some embodiments of FIG. 14 and FIG. 15, when the second clamping portion 310-2 deflects in the direction D1 toward the central axis B-B of the clip base 320 and abuts against the limiting portion 323, the second clamping portion 310-2 stops deflecting, thereby preventing the second clamping portion 310-2 from deflecting excessively and causing clamping failure.

In operation III, as shown in FIG. 16A and FIG. 16B, the clamping portion 310 transitions from the open state to the closed state to clamp the tissue 400.

If the second clamping portion 310-2 abuts against the limiting portion 323, the first control wire 110-1 is controlled by the control handle 120 to move from the distal end to the proximal end, causing the first clamping portion 310-1 to deflect in the direction toward the central axis B-B of the clip base 320 until the first clamping portion 310-1 and the second clamping portion 310-2 close.

If the second clamping portion 310-2 does not abut against the limiting portion 323, the first control wire 110-1 and the second control wire 110-2 are controlled by the control handle 120 to move from the distal end to the proximal end, causing the first clamping portion 310-1 and the second clamping portion 310-2 to deflect in the direction toward the central axis B-B of the clip base 320 until the first clamping portion 310-1 and the second clamping portion 310-2 close.

When the first clamping portion 310-1 and the second clamping portion 310-2 close, the distal end of the first clamping portion 310-1 and the distal end of the second clamping portion 310-2 may receive a portion of the tissue 400 into the clamping space, thereby closing the wound 410.

In operation IV, as shown in FIG. 17A and FIG. 17B, after the clamping portion 310 closes, the control wire 110 continues to move from the distal end to the proximal end, causing the locked portion 315 of the clamping portion 310 to extend axially to cooperate with the locking portion 326 of the clip base 320, and the clamping portion 310 is locked in the clip base 320. In some embodiments, the control wire 110 moves from the distal end to the proximal end, causing a portion of the hook 313 of each of the two clamping portions to deform and increase its axial length, so that the hook 313 extends into interior of the clip base 320 and cooperates with the locking portion 326 formed by the clip base 320, and the clamping portion 310 is locked in the clip base 320.

After the clamping portion 310 is locked, the control wire 110 continues to move from the distal end to the proximal end, releasing a connection between the control wire 110 and the clamping portion 310. In some embodiments, the control wire 110 moves from the distal end to the proximal end, causing the hook 313 to deform, displace, or break. Consequently, a connection between a hook 111 and the hook 313 is released, and the connection between the control wire 110 and the clamping portion 310 is released.

In operation V, as shown in FIG. 18A and FIG. 18B, after the connection between the control wire 110 and the clamping portion 310 is released, the control wire 110 continues to move from the distal end to the proximal end. After the hook 111 at the distal end of the control wire 110 cooperates with the connection pin 230, the hook 111 drives the bent portion 232 at the distal end of the connection pin 230 to disengage from the connection hole 325 of the clip base 320 and the connection recess of the sheath 210. The connection between the clip base 320 and the sheath 210 is released. At this time, the connection between the clip device 300 and the delivery device 200 is released.

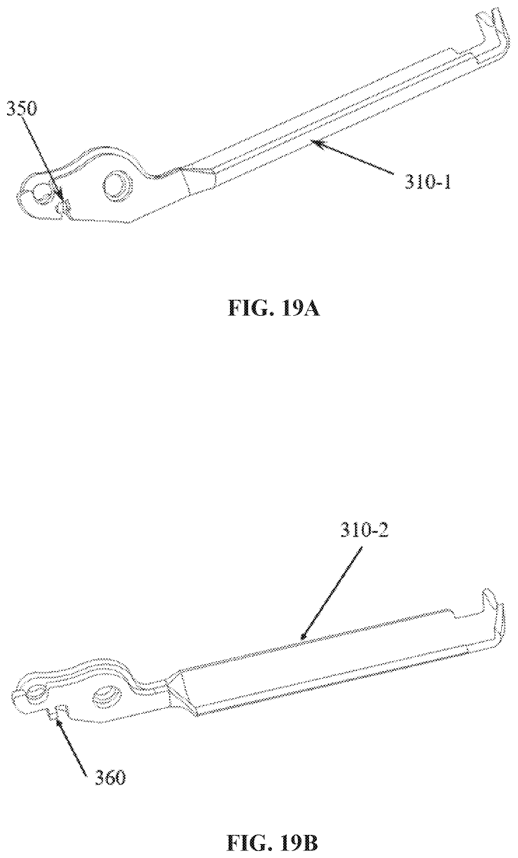

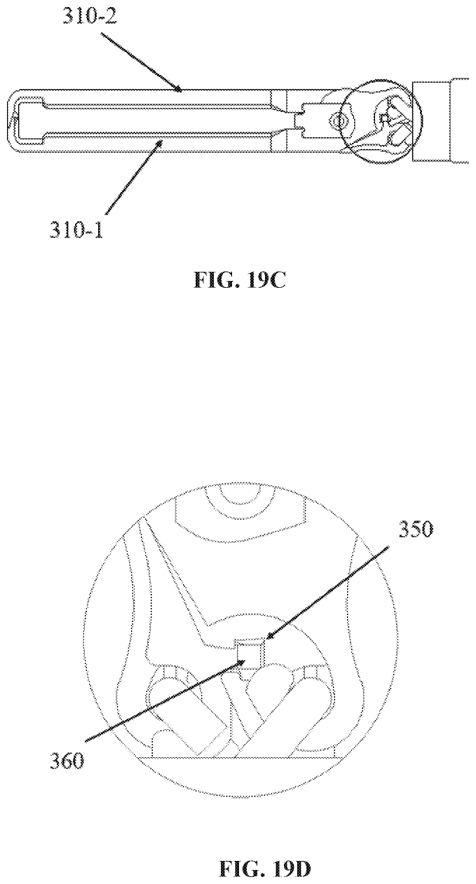

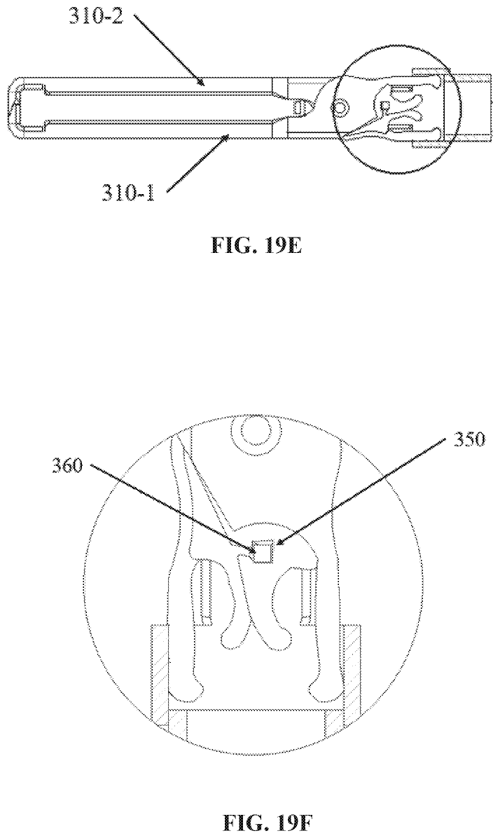

FIG. 19A is a schematic structural diagram of a first clamping portion according to some embodiments of the present disclosure. FIG. 19B is a schematic structural diagram of a second clamping portion according to some embodiments of the present disclosure. FIG. 19C is a schematic diagram of a closed state of a clip device according to some embodiments of the present disclosure. FIG. 19D is a partially enlarged schematic diagram of the clip device according to some embodiments of FIG. 19C. FIG. 19E is a schematic diagram of a released state of the clip device according to some embodiments of the present disclosure. FIG. 19F is a partially enlarged schematic diagram of the clip device according to some embodiments of FIG. 19E.

In some embodiments, one of the first clamping portion 310-1 and the second clamping portion 310-2 includes a locking portion 350, and the other of the first clamping portion 310-1 and the second clamping portion 310-2 includes a locked portion 360. The locking portion 350 and the locked portion 360 are respectively disposed on sides of the two clamping portions that face each other. The locking portion 350 includes a locked state and a released state. In some embodiments, when the clip device 300 is connected to the control wire 110, the locking portion 350 and the locked portion 360 are capable of relative movement, and the locking portion 350 is in the released state. When a connection between the clip device 300 and the control wire 110 is released, the locking portion 350 and the locked portion 360 are in a snap-fit connection, and the locking portion 350 is in the locked state.

For example, as shown in FIG. 19A and FIG. 19B, the first clamping portion 310-1 includes the locking portion 350. The locking portion 350 may be groove-shaped. The second clamping portion 310-2 includes the locked portion 360. The locked portion 360 may be a protrusion-shaped structure that matches the groove-shaped locking portion 350. The protrusion-shaped locked portion 360 may be inserted into the groove-shaped locking portion 350.

In some embodiments, as shown in FIG. 19C and FIG. 19D, when the clip device 300 is connected to the control wire 110, the clip instrument 10 is capable of performing operations such as opening, deflecting integrally, and closing through the control wire 110. At this time, the locking portion 350 is in the released state, that is, the protrusion-shaped locked portion 360 may be inserted into the groove-shaped locking portion 350 or withdrawn from the groove-shaped locking portion 350.

In some embodiments, as shown in FIG. 19E and FIG. 19F, when the connection between the clip device 300 and the control wire 110 is released, the locking portion is in the locked state, that is, the protrusion-shaped locked portion 360 may be snap-fit connected within the groove-shaped locking portion 350, so that the first clamping portion 310-1 and the second clamping portion 310-2 are locked to each other and not capable of being opened again.

In some embodiments, the locking portion 350 includes a locking space (e.g., a groove, a through hole, or a blind hole) for accommodating the locked portion 360. In some embodiments, when the clip device 300 is connected to the control wire 110, an opening size of the locking space is greater than a size of the locked portion 360. Therefore, the locked portion 360 may enter and withdraw from the locking portion 350 without obstruction, and no interference occurs between the locked portion 360 and the locking portion 350, which ensures that the clip instrument 10 can freely open and close within a preset range without hindrance. When the connection between the clip device 300 and the control wire 110 is released, the opening size of the locking space is less than the size of the locked portion 360. Therefore, the locked portion 360 is snap-fitted in the locking space, and the locked portion 360 and the locking portion 350 are not capable of relative movement, thereby keeping the clip instrument 10 in the closed state.

In some embodiments, when the locking portion 350 and the locked portion 360 are snap-fitted, surfaces of the locking portion 350 and the locked portion 360 that contact each other may be configured as matching inclined surfaces or smooth curved surfaces. The inclined surfaces or the smooth curved surfaces can serve as a guiding function, thereby guiding the relative movement between the locking portion 350 and the locked portion 360.

In some embodiments, as shown in FIG. 19F, during a release process of the clip device 300, the first control wire 110-1 and the second control wire 110-2 move toward the proximal end. The proximal ends of the first clamping portion 310-1 and the second clamping portion 310-2 are deformed under the action of an external force. During the deformation process, a gap between the locking portion 350 on the first clamping portion 310-1 and the locked portion 360 on the second clamping portion 310-2 gradually decreases, that is, the opening size of the locking space of the locking portion 350 gradually becomes smaller. When the deformation reaches a certain degree, the gap completely disappears. At this time, the opening size of the locking space may be considered to be less than the size of the locked portion 360, and the locking portion 350 and the locked portion 360 are tightly snap-fitted, thereby forming a stable locked state and effectively preventing further movement or loosening of the clamping portion 310.

By providing a locking portion and a locked portion, during use of the clip instrument, the clamping portions do not easily separate even when subjected to the external force, thereby improving stability and reliability of the clip instrument. Through the tight cooperation between the locking portion and the locked portion, the two clamping portions can maintain a fixed position in a pre-closed state or a fully closed state, which on one hand, strengthens a clamping force of the clip instrument, and on the other hand, avoids safety hazards caused by accidental opening of the clip instrument.

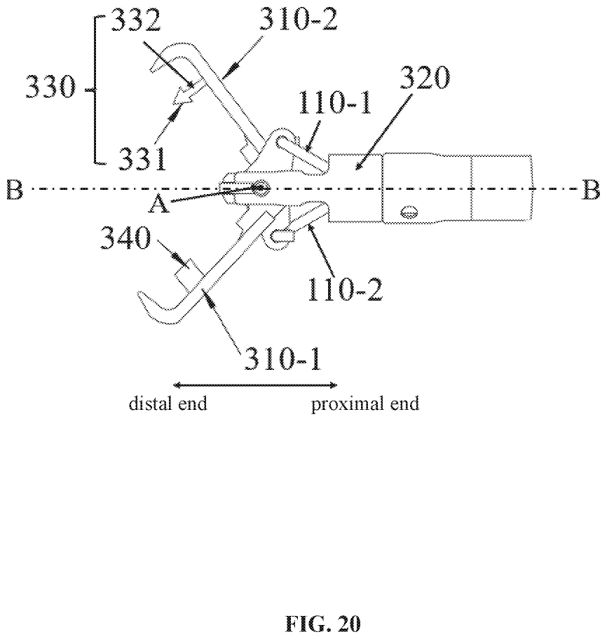

FIG. 20 is a schematic structural diagram of the clip device 300 according to some other embodiments of the present disclosure.

In some embodiments, at least one of the first clamping portion 310-1 or the second clamping portion 310-2 includes a puncture member 330. The puncture member 330 is a structure for enhancing a clamping force through puncture. In some embodiments, the puncture member 330 includes a puncture rod 332 and a puncture head 331 that are connected to each other. The puncture head 331 is connected to at least one of the first clamping portion 310-1 and the second clamping portion 310-2 through the puncture rod 332. During a process in which the clip device 300 switches from an open state to a closed state, the puncture head 331 may contact the tissue clamped by the clip device 300 before the clip device 300 closes and puncture into the tissue, which enhances a firm connection between the clip device 300 and the tissue, and avoids slippage of the clamped tissue when the clip device 300 closes and deflects.

In some embodiments, the puncture head 331 includes a hooking portion facing the puncture rod 332. For example, the hooking portion may be a barb or a barbed structure. The hooking portion is configured to hook the tissue from a direction toward the puncture rod 332, preventing the tissue from slipping off the puncture member 330, thereby further enhancing the clamping force of the clip device 300.

In some embodiments, for any puncture member 330, an end of the puncture rod 332 away from the puncture head 331 is connected to one of the two clamping portions. The puncture head 331 further includes a piercing portion. The piercing portion faces the other of the two clamping portions. For example, the piercing portion may be disposed on the first clamping portion 310-1. The piercing portion has a sharp end, and the sharp end faces the second clamping portion 310-2.

In some embodiments, the piercing portion includes a tip and a curved surface extending from the tip to the hooking portion. In some embodiments, the piercing portion includes the tip and a plurality of inclined surfaces extending from the tip to the hooking portion. With such a configuration, the piercing portion and the hooking portion can be smoothly connected, thereby improving a puncture effect. Meanwhile, because a contact surface area with the tissue is increased, penetration is facilitated, and the force can be distributed more evenly, thereby reducing a risk of tissue tearing.

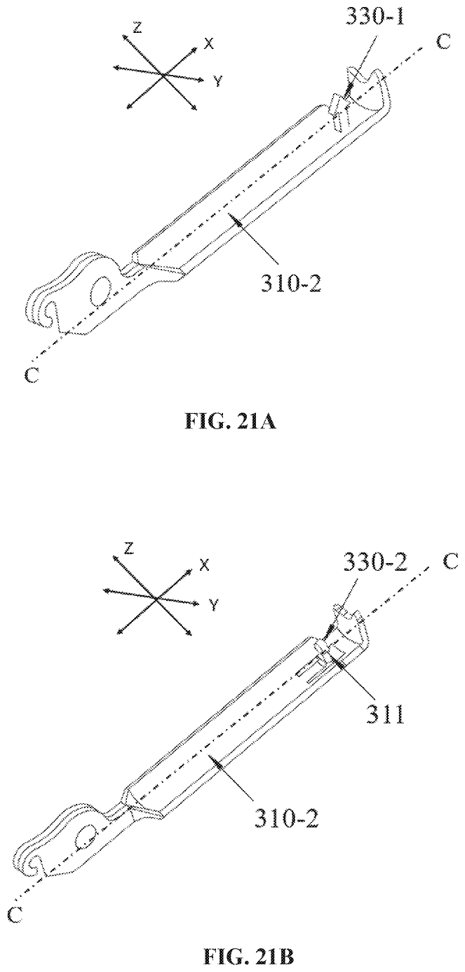

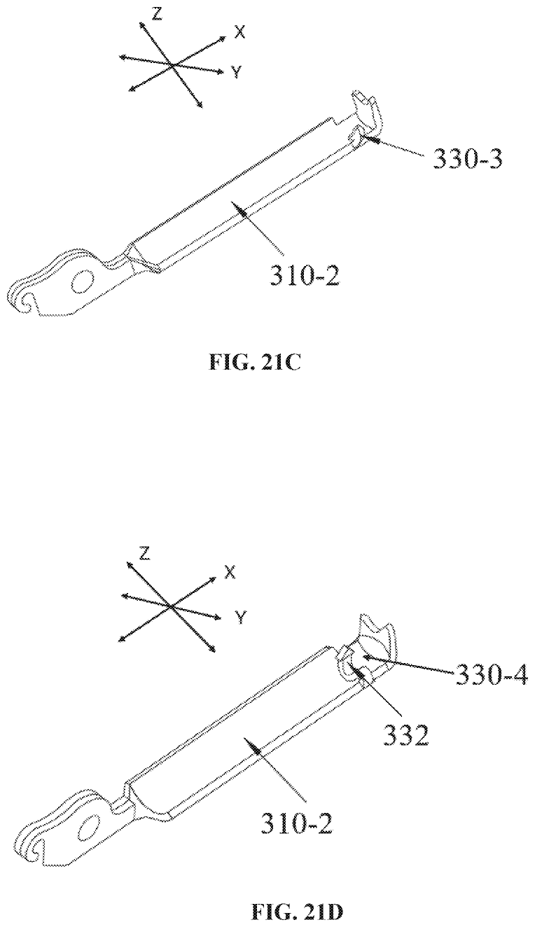

FIG. 21A is a schematic structural diagram of the puncture member 330 according to some embodiments of the present disclosure. FIG. 21B is a schematic structural diagram of the puncture member 330 according to some other embodiments of the present disclosure. FIG. 21C is a schematic structural diagram of the puncture member 330 according to another embodiments of the present disclosure. FIG. 21D is a schematic structural diagram of the puncture member 330 according to yet other embodiments of the present disclosure.

In some embodiments, the puncture member 330 may be disposed close to an axis C-C of the clamping portion 310, that is, the puncture member 330 is disposed near the axis C-C at a middle region of the clamping portion 310. In some embodiments, the puncture member 330 may be disposed on a side edge of the clamping portion 310.

In some embodiments, as shown in FIG. 21A and FIG. 21B, on the same projection plane, a projection of the puncture head 331 along a first direction Z is located in a first region of a projection of the clamping portion 310 (e.g., the second clamping portion 310-2) along the first direction Z. The first region is a middle region in a second direction Y of the projection of the clamping portion 310 (e.g., the second clamping portion 310-2) along the first direction Z. The second direction Y is a short axis direction of the clamping portion 310 (e.g., the second clamping portion 310-2). The first direction Z is perpendicular to the short axis direction and a long axis direction of the clamping portion 310 (e.g., the second clamping portion 310-2). The long axis direction may be denoted as a third direction X. The middle region refers to a preset range region on both sides of the axis C-C of the clamping portion 310 (e.g., the second clamping portion 310-2). For example, a width of the preset range region in the second direction Y accounts for 30% to 50% of a width of the clamping portion 310 in the second direction Y.

In some embodiments, an end of the puncture rod 332 away from the puncture head 331 is connected to the middle region of the clamping portion 310 (e.g., the second clamping portion 310-2) along the second direction Y. It should be noted that the middle region to which the puncture rod 332 is connected is the middle region of the clamping portion 310, not the middle region of the projection of the clamping portion 310 mentioned above.

In some embodiments, as shown in FIG. 21C and FIG. 21D, the end of the puncture rod 332 away from the puncture head 331 is connected to a side region of the clamping portion 310 (e.g., the second clamping portion 310-2) along the second direction Y. The side region refers to a preset region on two side edges of the clamping portion 310 distributed along the second direction Y. The preset region may extend along the third direction X. A length of the preset region in the third direction X accounts for 30% to 50% of a length of the clamping portion 310 in the third direction X. The preset region may be disposed closer to the distal end of the clamping portion 310. In some embodiments, the puncture rod 332 may be configured as a curved shape (e.g., a curved shape including at least one bent portion). The puncture rod 332 may deform moderately to better conform to a tissue contour. More description may be found in the description of FIG. 21D.

In some embodiments, on the same projection plane, the projection of the puncture head 331 along the first direction Z is located in a second region of the projection of the clamping portion 310 (e.g., the second clamping portion 310-2) along the first direction Z. The second region is a side region in the second direction Y of the projection of the clamping portion 310 (e.g., the second clamping portion 310-2) along the first direction Z. The second direction Y is the short-axis direction of the clamping portion, and the first direction Z is perpendicular to the short-axis direction and the long-axis direction of the clamping portion. The long-axis direction may be denoted as the third direction X. More description regarding the side region may be found in the description above.

In some embodiments, the end of the puncture rod 332 away from the puncture head 331 is connected to the side region of the clamping portion 310 along the second direction Y. Therefore, the puncture member 330 can be manufactured independently and then connected to the clamping portion 310, facilitating processing and manufacturing.

In some embodiments, as shown in FIG. 21A, a puncture member 330-1 is disposed on an inner side of the clamping portion 310 (e.g., the second clamping portion 310-2) and near the distal end of the clamping portion 310. The puncture head 331 includes two hooking portions arranged symmetrically, so that the puncture head 331 has an arrow shape. The hooking portions are distributed along the axis C-C of the clamping portion 310. In some embodiments, the puncture member 330-1 may further include a plurality of hooking portions. The plurality of hooking portions may be uniformly or non-uniformly distributed circumferentially around the puncture rod 332, so that an end of the puncture member 330-1 has a pyramid shape.

In some embodiments, the puncture member 330-1 may be processed by a laser cutting machine, resulting in high forming stability. The second clamping portion 310-2 may be formed by stamping, and a stamping hole is punched near the distal end of the second clamping portion 310-2. The puncture member 330-1 may be welded as one piece with the stamping hole.

In some embodiments, the puncture member 330-1 may be manufactured independently and then connected to the clamping portion 310, facilitating processing. Once the puncture member 330-1 penetrates into the tissue, the structure of the hooking portion can more effectively engage with the tissue, providing a stronger fixation force than a needle-shaped puncture device and reducing a risk of postoperative displacement.

In some embodiments, as shown in FIG. 21B, a puncture member 330-2 is disposed on the inner side of the clamping portion 310 (e.g., the second clamping portion 310-2) and near the distal end of the clamping portion 310, and the puncture member 330 is disposed close to the side edge of the clamping portion 310. The puncture head 331 includes two hooking portions arranged symmetrically, so that the puncture head 331 has an arrow shape. The hooking portions are distributed along a direction perpendicular to the direction of the axis C-C of the clamping portion 310. In some embodiments, the puncture member 330-2 may also include a plurality of hooking portions. The plurality of hooking portions may be distributed uniformly or non-uniformly along a circumference of the puncture rod 332.

In some embodiments, an external contour of the puncture member 330-2 may be formed on the second clamping portion 310-2 by stamping first, and an angle of the puncture member 330-2 may then be bent from 180 degrees in a plane to 90 degrees by a bending tool.

In some embodiments, the puncture member 330-2 may be streamlined, and has a sharp front end to ensure good penetration. Meanwhile, a side profile of the puncture member 330-2 is a relatively smooth curve to reduce resistance and tissue damage during insertion. The streamlined puncture member 330-2 can better disperse forces, reduce local pressure, and improve patient comfort through optimized mechanical design.

In some embodiments, as shown in FIG. 21C, a puncture member 330-3 is disposed on a side of the clamping portion 310, thereby facilitating processing and manufacturing. The puncture head 331 may include a single hooking portion, such that the puncture head 331 has a barb shape. In some embodiments, the puncture member 330-3 may also include two or more hooking portions arranged symmetrically, such that the puncture member 330-3 has an arrow shape or a pyramid shape. The two or more hooking portions may be distributed along the axis C-C direction of the clamping portion 310 or other directions. For example, the two or more hooking portions may be distributed uniformly or non-uniformly along the circumference of the puncture rod 332.

In some embodiments, a shape of the puncture member 330-3 may be reserved on the second clamping portion 310-2. An angle of the puncture member 330-3 may then be bent from 180 degrees in a plane to 90 degrees by a bending tool.

In some embodiments, the puncture member 330-3 is formed on a side of the second clamping portion 310-2 and has a triangular pyramid shape, with three inclined surfaces converging to a tip, which increases a surface area in contact with the tissue, while the tip facilitates penetration. The triangular pyramid structure can distribute the force more evenly during fixation, reducing a risk of tissue tearing.

In some embodiments, as shown in FIG. 21D, a puncture member 330-4 is disposed on a side of the clamping portion 310. The puncture rod 332 may have a bent shape, thereby forming a hooking portion.

In some embodiments, a shape of the puncture member 330-4 is reserved on the second clamping portion 310-2. An angle of the puncture member 330-4 is then bent from 180 degrees in a plane to 90 degrees by a bending tool. Finally, the puncture member 330-4 is corrected by a correction tool to make the puncture member 330-4 adhere to a contour of the second clamping portion 310-2.

In some embodiments, the puncture member 330-4 with a bent structure may unfold along a side wall of the second clamping portion 310-2, providing a larger contact area with the tissue. Meanwhile, according to different thicknesses or elasticity of the tissue, the puncture member 330-4 with the bent structure can deform appropriately to better conform to a tissue contour.

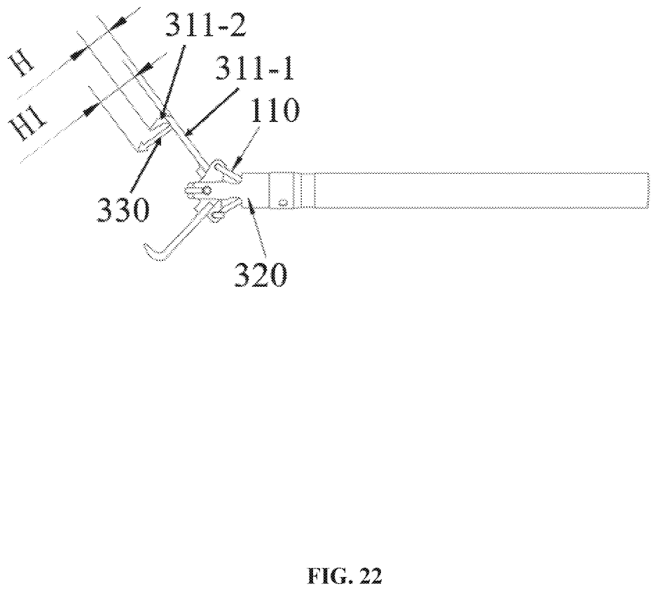

FIG. 22 is a schematic diagram of a height relationship between the puncture member 330 and a first clip head 311-2 according to some embodiments of the present disclosure.

In some embodiments, as shown in FIG. 22, the first clamping portion 310-1 includes a first clip arm 311-1 and a first clip head 311-2 connected to each other. The first clip head 311-2 is disposed at a distal end of the first clip arm 311-1. A free end of the first clip head 311-2 extends radially inward of the clip device 300. The puncture member 330 is disposed on the first clip arm 311-1. In some embodiments, a ratio of a height H1 of the puncture member 330 to a height H of the first clip head 311-2 ranges from 1.2 to 3. In some embodiments, the ratio of the height H1 of the puncture member 330 to the height H of the first clip head 311-2 ranges from 1.5 to 2.5. In some embodiments, the ratio of the height H1 of the puncture member 330 to the height H of the first clip head 311-2 ranges from 1.8 to 2. H1 is a distance from an end of the puncture member 330 to an inner surface of the first clamping portion 310-1. H is a distance from an end of the first clip head 311-2 to the inner surface of the first clamping portion 310-1. In some embodiments, the second clamping portion 310-2 may include a second clip arm and a second clip head connected to each other. A free end of the second clip head extends radially inward of the clip device. The puncture member 330 is disposed on the second clip arm. In some embodiments, a ratio of a height of the puncture member 330 to a height of the second clip head ranges from 1.2 to 3. In some embodiments, the ratio of the height of the puncture member 330 to the height of the second clip head ranges from 1.5 to 2.5. In some embodiments, the ratio of the height of the puncture member 330 to the height of the second clip head ranges from 1.8 to 2. Setting the puncture member 330 and the first clip head 311-2 according to the ratio relationship between the height of the puncture member 330 and the height of the first clip head 311-2 can effectively reduce a risk of tissue slippage when the clip device 300 closes to clamp tissue.

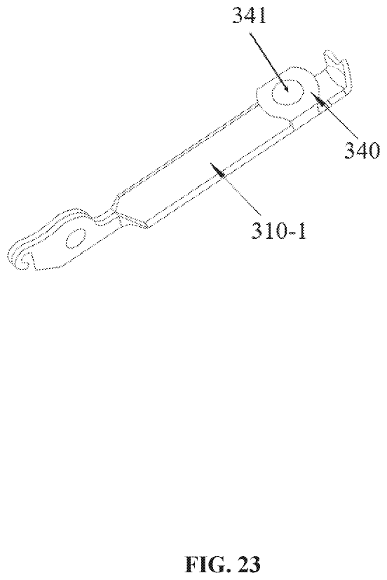

FIG. 23 is a schematic structural diagram of a puncture platform 340 according to some embodiments of the present disclosure.

In some embodiments, as shown in FIG. 20, at least one of the first clamping portion 310-1 or the second clamping portion 310-2 (e.g., the first clamping portion 310-1) includes a puncture platform 340. The puncture platform 340 is a structure that cooperates with the puncture member 330. The puncture platform 340 may provide a support platform for the tissue before the puncture member 330 performs puncture. Meanwhile, the puncture platform 340 provides a fulcrum for the puncture member 330 and has a certain positioning function to facilitate the puncture member 330 smoothly puncturing into the tissue. In some embodiments, a surface of the puncture platform 340 may be flat or convex to facilitate providing support for the tissue.

In some embodiments, one of the first clamping portion 310-1 and the second clamping portion 310-2 may be provided with one puncture member 330, and the other of the first clamping portion 310-1 and the second clamping portion 310-2 may be provided with one puncture platform 340. In some embodiments, the first clamping portion 310-1 is provided with one puncture member 330 and one puncture platform 340, and the second clamping portion 310-2 is also provided with another puncture member 330 and another puncture platform 340.

For example, the first clamping portion 310-1 is provided with a first puncture member, and the first clamping portion 310-1 is provided with a first puncture platform. The second clamping portion 310-2 is provided with a second puncture platform, and the second clamping portion 310-2 is provided with a second puncture member. When the clip device 300 is in the closed state, a position of the first puncture member on the first clamping portion 310-1 may coincide with a position of the second puncture platform on the second clamping portion 310-2. Meanwhile, a position of the first puncture platform on the first clamping portion 310-1 may coincide with a position of the second puncture member on the second clamping portion 310-2. That is, the first puncture member cooperates with the second puncture platform, thereby stably connecting the first clamping portion 310-1 and the second clamping portion 310-2 to the tissue at the coincided position. Similarly, the second puncture member cooperates with the first puncture platform, thereby stably connecting the first clamping portion 310-1 and the second clamping portion 310-2 to the tissue at the coincided position.

In some embodiments, as shown in FIG. 23, the puncture platform 340 is provided with a puncture space 341. When the first clamping portion 310-1 and the second clamping portion 310-2 close, the puncture member 330 may penetrate into the puncture space 341. When the first clamping portion 310-1 and the second clamping portion 310-2 open, the puncture member 330 may withdraw from the puncture space 341. In some embodiments, the puncture space 341 may be a through hole or a blind hole provided on the puncture platform 340.

In some embodiments, an outer diameter of the puncture member 330 may be less than an inlet diameter of the puncture space 341. That is, the outer diameter of the puncture member 330 may be less than an inner diameter of the through hole or the blind hole provided on the puncture platform 340, to facilitate the puncture member 330 smoothly puncturing through the tissue. It should be noted that the puncture member 330 and the puncture space 341 do not lock to each other. The puncture member 330 only needs to pass through the puncture space 341.

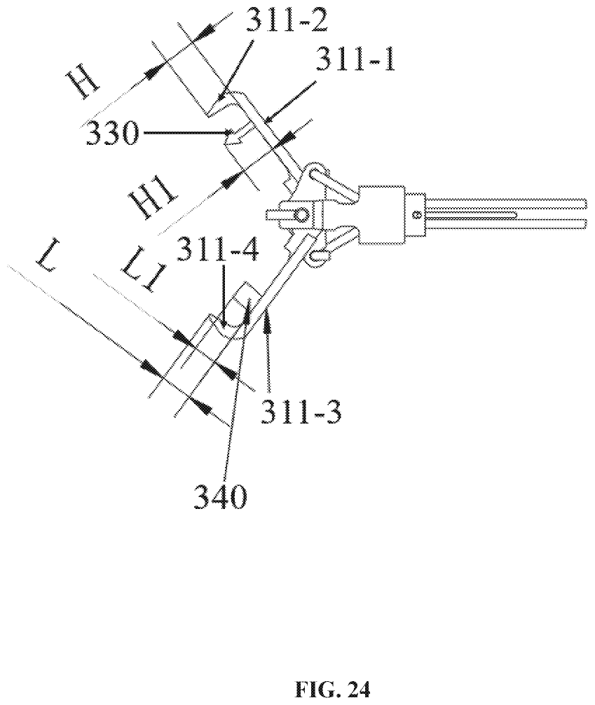

FIG. 24 is a schematic diagram of height relationships between the puncture member 330 and the first clip head 311-2 and between the puncture platform 340 and a second clip head 311-4 according to some embodiments of the present disclosure.

In some embodiments, the first clamping portion 310-1 includes the first clip arm 311-1 and the first clip head 311-2 connected to each other. The second clamping portion 310-2 includes a second clip arm 311-3 and the second clip head 311-4 connected to each other. Free ends of the first clip head 311-2 and the second clip head 311-4 extend radially inward of the clip device 300. In some embodiments, as shown in FIG. 24, the puncture member 330 is disposed on the first clip arm 311-1, and the puncture platform 340 is disposed on the second clip arm 311-3. A ratio of the height H1 of the puncture member 330 to a height H of the first clip head 311-2 ranges from 0.8 to 1.2. A ratio of a height L1 of the puncture platform 340 to a height L of the second clip head 311-4 ranges from 0.8 to 1.2. H1 is a distance from an end of the puncture member 330 to the inner surface of the first clamping portion 310-1. H is a distance from an end of the first clip head 311-2 to the inner surface of the first clamping portion 310-1. L1 is a distance from a surface of the puncture platform 340 to an inner surface of the second clamping portion 310-2. L is a distance from the second clip head 311-4 to the inner surface of the second clamping portion 310-2. In some embodiments, the puncture member 330 is disposed on the second clip arm 311-3, and the puncture platform 340 is disposed on the first clip arm 311-1. A ratio of a height of the puncture member 330 to a height of the second clip head 311-4 ranges from 0.8 to 1.2. A ratio of a height of the puncture platform 340 to a height of the first clip head 311-2 ranges from 0.8 to 1.2. Setting the puncture member 330, the first clip head 311-2, the puncture platform 340, and the second clip head 311-4 according to the aforementioned height ratio relationships can effectively reduce a risk of tissue slippage when the clip device 300 closes to clamp the tissue.

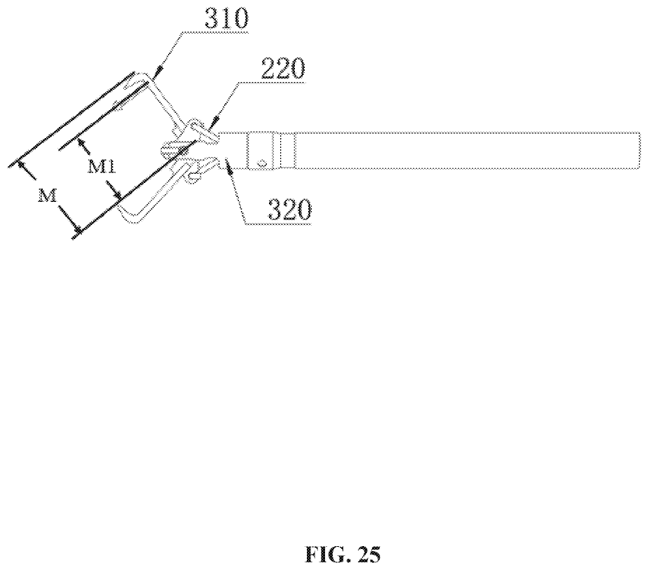

FIG. 25 is a schematic diagram of a length relationship between the puncture member 330 and the clamping portion 310 according to some embodiments of the present disclosure.

In some embodiments, as shown in FIG. 25, a ratio of a distance M1 between the puncture member 330 and the proximal end of the clamping portion 310 to a length M of the clamping portion 310 ranges from 0.6 to 0.9. M1 is a distance from a center line of the puncture member 330 to the proximal end of the clamping portion 310. M is a distance from the distal end of the clamping portion to the proximal end of the clamping portion. Setting the position of the puncture member 330 relative to the clamping portion 310 according to the above distance range can better achieve a puncture effect of the puncture member 330 and improve clamping force.

Some embodiments of the present disclosure further provide a control method for a clip instrument. The control method is applied to the clip instrument 10 according to any one of the foregoing embodiments. The clip instrument includes the clip device 300, the delivery device 200, and the control device 100. The clip device 300 is releasably disposed at the distal end of the delivery device 200. The control device 100 includes the control wire 110. The control wire 110 includes the first control wire 110-1 and the second control wire 110-2. The clip device 300 includes the clip base 320 and the clamping portion 310. The clamping portion 310 includes the first clamping portion 310-1 and the second clamping portion 310-2. The distal end of the first control wire 110-1 is releasably connected to the first clamping portion 310-1. The distal end of the second control wire 110-2 is releasably connected to the second clamping portion 310-2. In some embodiments, the control method is performed by the control device 100. The operations of the following processes are for illustrative purposes only. In some embodiments, the processes may be completed with one or more additional operations not described and/or without one or more of the operations discussed. Furthermore, an order of the operations of the processes described below is not intended to be limiting.

In some embodiments, the control method includes controlling the first control wire 110-1 to move from the proximal end to the distal end by the control device 100 to cause the first control wire 110-1 to drive the first clamping portion 310-1 to deflect around an axis.

In some embodiments, the control device 100 includes the two control handles 120. The distal end of the first control wire 110-1 is connected to the proximal end of the first clamping portion 310-1. When the first control handle 120-1 controls the first control wire 110-1 to move from the proximal end to the distal end, the proximal end of the first clamping portion 310-1 receives a pushing force, causing the first clamping portion 310-1 to deflect around an axis of the pin 321 in the direction D2 away from the central axis B-B of the clip base 320.

In some embodiments, the control method includes controlling the second control wire 110-2 to move from the distal end to the proximal end by the control device 100 to cause the second control wire 110-2 to drive the second clamping portion 310-2 to deflect around the axis A. The deflection direction of the first clamping portion 310-1 and the deflection direction of the second clamping portion 310-2 are the same, and the clip device 300 deflects integrally.

In some embodiments, the distal end of the second control wire 110-2 is connected to the proximal end of the second clamping portion 310-2. When the second control handle 120-2 controls the second control wire 110-2 to move from the distal end to the proximal end, the proximal end of the second clamping portion 310-2 receives a pulling force, causing the second clamping portion 310-2 to deflect around the axis of the pin 321 in the direction D1 toward the central axis B-B of the clip base 320.

The first clamping portion 310-1 deflects in the direction D2 away from the central axis B-B of the clip base 320, and the second clamping portion 310-2 deflects in the direction D1 toward the central axis B-B of the clip base 320. The deflection directions of the first clamping portion 310-1 and the second clamping portion 310-2 are the same, thereby achieving an integral deflection of the clamping portion 310. A clamping direction of the integral-deflected clamping portion 310 can directly face the wound 410, achieving effective clamping.

Some embodiments of the present disclosure further provide a control method for a clip instrument. The control method is applied to the clip instrument 10 according to any one of the foregoing embodiments. The clip instrument includes the clip device 300, the delivery device 200, and the control device 100. The clip device 300 is releasably disposed at the distal end of the delivery device 200. The control device 100 includes the control wire 110. The control wire 110 includes the first control wire 110-1 and the second control wire 110-2. The clip device 300 includes the clip base 320 and the clamping portion 310. The clamping portion 310 includes the first clamping portion 310-1 and the second clamping portion 310-2. The distal end of the first control wire 110-1 is releasably connected to the first clamping portion 310-1. The distal end of the second control wire 110-2 is releasably connected to the second clamping portion 310-2. In some embodiments, the control method is performed by the control device 100. The operations of the following processes are for illustrative purposes only. In some embodiments, the process may be completed with one or more additional operations not described and/or without one or more of the operations discussed. Furthermore, the order of the operations of the processes described below is not intended to be limiting.

In some embodiments, the control method for the clip instrument includes controlling the first control wire 110-1 to move toward the distal end by the control device 100 to cause the clip instrument 10 to be in the side-open state. More description regarding the process for controlling the clip instrument 10 to be in the side-open state by controlling the first control wire 110-1 to move toward the distal end may be found in related description of FIG. 13-FIG. 15 above.

In some embodiments, the control method for the clip instrument further includes continuing to control the first control wire 110-1 to move toward the distal end when a side-opening angle of the clip instrument 10 reaches a maximum value, where the first clamping portion 310-1 and the second clamping portion 310-2 deflect in the same direction.

In some embodiments, when the side-opening angle of the clip instrument 10 reaches the maximum value, continuing to control the first control wire 110-1 to move toward the distal end to drive the first clamping portion 310-1 to deflect in the direction D2 away from the central axis B-B of the clip base 320, can cause the second control wire 110-2 to deform and drive the second clamping portion 310-2 to deflect in the direction D1 toward the central axis B-B of the clip base 320, thereby achieving deflection of the first clamping portion 310-1 and the second clamping portion 310-2 in the same direction. The second control wire 110-2 deforms by abutting against the first clamping portion 310-1. More description may be found in the related description of FIG. 7-FIG. 8C.

In some embodiments, the control method further includes controlling the first clamping portion 310-1 to deflect around the axis A in the direction D2 away from the central axis B-B of the clip base 320 to a position where the first clamping portion 310-1 abuts against the second control wire 110-2 or the second clamping portion 310-2.

In some embodiments, the control device 100 includes one or two control handles 120. The control device 100 adjusts a position of the clip device 300 by adjusting a position of the delivery device or adjusting a position of the sheath, so that one of the two clamping portions of the clip device 300 abuts against an edge of the wound 410 of the tissue 400. When the distal end of the first clamping portion 310-1 abuts against the tissue 400, by controlling the sheath 210 or the clamping portion 310 to move toward the distal end, the distal end of the first clamping portion 310-1 receives a pressure and deflects in the direction D2 away from the central axis B-B of the clip base 320. The control device 100 continuously controls the first clamping portion 310-1 to deflect until the first clamping portion 310-1 deflects to the position where the first clamping portion 310-1 abuts against the second control wire 110-2 or the second clamping portion 310-2. At this time, the contact portion 314 of the first clamping portion 310-1 abuts against the mating portion 112 of the second control wire 110-2, or the contact portion 314 of the first clamping portion 310-1 abuts against the mating portion 112 of the second clamping portion 310-2.

In some embodiments, the control method for the clip instrument further includes controlling the first clamping portion 310-1 to continue to deflect around the axis A in the direction D2 away from the central axis B-B of the clip base 320, and driving the second clamping portion 310-2 to deflect around the axis A while causing the second control wire 110-2 to deform.

In some embodiments, when the first clamping portion 310-1 deflects in the direction D2 away from the central axis B-B of the clip base 320, the contact portion 314 of the first clamping portion 310-1 abuts against the mating portion 112 of the second control wire 110-2. The first clamping portion 310-1 continues to deflect in the direction D2 away from the central axis B-B of the clip base 320, causing at least a portion of the distal end of the second control wire 110-2 to be squeezed and deform. After the second control wire 110-2 deforms, the second control wire 110-2 drives the second clamping portion 310-2 to deflect in the direction D1 toward the central axis B-B of the clip base 320.

In some embodiments, when the first clamping portion 310-1 deflects in the direction D2 away from the central axis B-B of the clip base 320, the contact portion 314 of the first clamping portion 310-1 abuts against the mating portion 112 of the second clamping portion 310-2. The first clamping portion 310-1 continues to deflect in the direction D2 away from the central axis B-B of the clip base 320, driving the second clamping portion 310-2 to deflect in the direction D1 toward the central axis B-B of the clip base 320. When the second clamping portion 310-2 deflects, at least a portion of the distal end of the second control wire 110-2 is squeezed and deforms.

The first clamping portion 310-1 deflects in the direction D2 away from the central axis B-B of the clip base 320, and the second clamping portion 310-2 deflects in the direction D1 toward the central axis B-B of the clip base 320. The deflection directions of the first clamping portion 310-1 and the second clamping portion 310-2 are the same, thereby achieving an integral deflection of the clamping portion 310. The clamping direction of the integral-deflected clamping portion 310 can directly face the wound 410, achieving effective clamping.

In some embodiments, the control method further includes controlling the first control wire 110-1 to move from the proximal end to the distal end by the control device 100 to cause the first control wire 110-1 to drive the first clamping portion 310-1 to deflect around an axis.

In some embodiments, the first control handle 120-1 is connected to the proximal end of the first control wire 110-1, and the first control wire 110-1 is connected to the first hook 313 of the first clamping portion 310-1. In some embodiments, after the clamping portion 310 is delivered to a target position (e.g., near the wound 410 of the tissue 400) through the delivery device 200, the first control handle 120-1 controls the first control wire 110-1 to move from the proximal end to the distal end to drive the first clamping portion 310-1 to deflect around the axis A in a direction away from the central axis B-B of the clip base 320.

In some embodiments, the control method further includes controlling the second control wire 110-2 to move from the proximal end to the distal end by the control device 100 to cause the second control wire 110-2 to drive the second clamping portion 310-2 to deflect around an axis. The first clamping portion 310-1 and the second clamping portion 310-2 deflect in directions away from each other, and the clip device 300 opens.

In some embodiments, the second control handle 120-2 is connected to the proximal end of the second control wire 110-2, and the second control wire 110-2 is connected to the second hook 313 of the second clamping portion 310-2. The second control wire 110-2 moves from the proximal end to the distal end to drive the second clamping portion 310-2 to deflect around the axis A in the direction away from the central axis B-B of the clip base 320. At this time, the first clamping portion 310-1 and the second clamping portion 310-2 are away from each other and are in the open state. The clamping space is formed between the first clamping portion 310-1 and the second clamping portion 310-2, and the wound 410 of the tissue 400 may be clamped in the clamping space.

In some embodiments, the control device 100 is further configured to control the clip device to perform operations such as closing, locking, and releasing. The following embodiments will describe the control method for closing, locking, and releasing the clip instrument.

In some embodiments, after the clamping portion 310 is opened or integral-deflected, the control handle 120 controls the first control wire 110-1 to move from the distal end to the proximal end, causing the first control wire 110-1 to drive the first clamping portion 310-1 to deflect around an axis. The control handle 120 controls the second control wire 110-2 to move from the distal end to the proximal end, causing the second control wire 110-2 to drive the second clamping portion 310-2 to deflect around the axis, the first clamping portion 310-1 and the second clamping portion 310-2 deflect in directions toward each other, and the clip device 300 closes.

In some embodiments, if the second clamping portion 310-2 abuts against the limiting portion 323, then the control handle 120 controls the first control wire 110-1 to move from the distal end to the proximal end, causing the first clamping portion 310-1 to deflect around the axis A in the direction toward the central axis B-B of the clip base 320 until the first clamping portion 310-1 and the second clamping portion 310-2 form a closure.

If the second clamping portion 310-2 does not abut against the limiting portion 323, then the control handle 120 controls the first control wire 110-1 and the second control wire 110-2 to move from the distal end to the proximal end, causing the first clamping portion 310-1 and the second clamping portion 310-2 to both deflect around the axis A in the direction toward the central axis B-B of the clip base 320 until the first clamping portion 310-1 and the second clamping portion 310-2 form the closure.

When the first clamping portion 310-1 and the second clamping portion 310-2 are closed, a portion of the tissue 400 may be clamped in the clamping space through the distal end of the first clamping portion 310-1 and the distal end of the second clamping portion 310-2, thereby closing the wound 410.

In some embodiments, after the clamping portion 310 is closed, the first control handle 120 controls the first control wire 110-1 to move from the distal end to the proximal end, causing the first control wire 110-1 to drive the first clamping portion 310-1 to form a limiting fit with the clip base 320, and the first control wire 110-1 is released from the first clamping portion 310-1. The second control handle 120-2 controls the second control wire 110-2 to move from the distal end to the proximal end, causing the second control wire 110-2 to drive the second clamping portion 310-2 to form a limiting fit with the clip base 320, and the second control wire 110-2 is released from the second clamping portion 310-2. The first clamping portion 310-1 and the second clamping portion 310-2 are both locked to the clip base 320, the clip device 300 is locked, and the control wire 110 is released from the clamping portion 310.

In some embodiments, after the clamping portion 310 is closed, the control wire 110 continues to move from the distal end to the proximal end, causing the locked portion 315 of the clamping portion 310 to extend axially to cooperate with the locking portion 326 of the clip base 320, and the clamping portion 310 is locked in the clip base 320. In some embodiments, the control wire 110 moves from the distal end to the proximal end, causing a portion of the hook 313 of each of the two clamping portions to deform to increase its axial length of the hook 313, so that the hook 313 extends into the clip base 320 and cooperates with the locking portion 326 formed in the clip base 320, and the clamping portion 310 is locked in the clip base 320.

After the clamping portion 310 is locked, the control wire 110 continues to move from the distal end to the proximal end, causing the connection between the control wire 110 and the clamping portion 310 to be released. In some embodiments, the control wire 110 moving from the distal end to the proximal end causes the hook 313 to deform, displace, or break, then the connection between the hook 111 and the hook 313 is disengaged, and the connection between the control wire 110 and the clamping portion 310 is released.

In some embodiments, the clip base 320 and the sheath 210 are releasably connected through the connection pin 230, controlling the control wire 110 to continue to move from the distal end to the proximal end causes the control wire 110 to drive the connection pin 230 to be released from the clip base 320 and the sheath 210, and the clip device 300 is released from the delivery device 200.

In some embodiments, after the connection between the control wire 110 and the clamping portion 310 is released, the control wire 110 continues to move from the distal end to the proximal end. After the hook 111 at the distal end of the control wire 110 cooperates with the connection pin 230, the hook 111 drives the bent portion 232 at the distal end of the connection pin 230 to disengage from the connection hole 325 of the clip base 320 and the connection recess of the sheath 210, and the connection between the clip base 320 and the sheath 210 is disengaged. At this time, the clip device 300 and the delivery device 200 are released from each other.

Some embodiments of the present disclosure further provide a control method for a clip instrument. The control method is applied to the clip instrument 10 according to any one of the foregoing embodiments. The clip instrument includes the clip device 300, the delivery device 200, and the control device 100. The clip device 300 is releasably disposed at the distal end of the delivery device 200. The control device 100 includes the control wire 110. The control wire 110 includes the first control wire 110-1 and the second control wire 110-2. The clip device 300 includes the clip base 320 and the clamping portion 310. The clamping portion 310 includes the first clamping portion 310-1 and the second clamping portion 310-2. The distal end of the first control wire 110-1 is releasably connected to the first clamping portion 310-1. The distal end of the second control wire 110-2 is releasably connected to the second clamping portion 310-2. At least one of the first clamping portion 310-1 or the second clamping portion 310-2 includes the puncture member 330. In some embodiments, the control method is performed by the control device 100.

In some embodiments, the control method for the clip instrument 10 includes: controlling the clip instrument 10 to clamp a first target object at a first target position and causing the puncture member 330 to penetrate into the first target object; controlling at least a portion of the clip device 300 to move to a second target position, wherein the puncture member 330 drives the first target object to move synchronously; and controlling the clip instrument 10 to simultaneously clamp the first target object and the second target object. The first target object and the second target object are both the tissue. However, because the tissue may have a large span, different positions of the tissue are respectively considered as the first target object and the second target object. That is, the tissue at the first target position may be considered as the first target object, and the tissue at the second target position may be considered as the second target object.

In some embodiments, controlling at least a portion of the clip device 300 to move to the second target position refers to moving the two clamping portions of the clip device 300 to the second target position, or moving one of the two clamping portions to the second target position, so that the clip instrument 10 may clamp the second target object when the clip instrument 10 closes at the second target position. In some embodiments, controlling at least a portion of the clip device 300 to move to the second target position may refer to moving the entire clip device 300 to the second target position. This scenario may correspond to a situation where the tissue span is extremely large, and movement of the clamping portion 310 alone may not reach the second target position, thus requiring movement of the entire clip device 300. In some embodiments, during a process of controlling at least a portion of the clip device 300 to move to the second target position, the puncture member 330 drives the first target object to move synchronously.

In some embodiments, controlling the clip instrument 10 to clamp the first target object at the first target position and causing the puncture member 330 to penetrate into the first target object includes: controlling the first control wire 110-1 and the second control wire 110-2 to move from the proximal end toward the distal end to cause the clip instrument 10 to be in the open state; and controlling the clip instrument 10 to move to the first target position; controlling the first control wire 110-1 and the second control wire 110-2 to move toward the proximal end to cause the clip instrument 10 to clamp the first target object.

In some embodiments, an initial state of the clip instrument 10 may be the closed state. Subsequently, the first control wire 110-1 and the second control wire 110-2 are controlled to move from the proximal end toward the distal end. The first control wire 110-1 drives the first clamping portion 310-1 to deflect in the direction D2 away from the central axis B-B of the clip base 320. The second control wire 110-2 drives the second clamping portion 310-2 to deflect in the direction away from the central axis B-B of the clip base 320, which is opposite to the direction D1. The clip instrument 10 opens. After controlling the clip instrument 10 to move to the first target position, the first control wire 110-1 is controlled to move toward the proximal end, driving the first clamping portion 310-1 to deflect opposite to the direction D2 toward the central axis B-B of the clip base 320. The second control wire 110-2 is controlled to move toward the proximal end, driving the second clamping portion 310-2 to deflect in the direction D1 toward the central axis B-B of the clip base 320. The clip instrument 10 closes, and the clip instrument 10 clamps the first target object.

In some embodiments, before controlling the clip instrument 10 to clamp the first target object at the first target position, the clip instrument 10 is controlled to switch to the side-open state. In some embodiments, after the puncture member 330 penetrates into the first target object, the clip instrument 10 is controlled to switch to the side-open state.

In some embodiments, controlling the clip instrument 10 to switch to the side-open state includes: controlling one of the first control wire 110-1 and the second control wire 110-2 to move toward the distal end. In some embodiments, controlling the clip instrument 10 to switch to the side-open state further includes: controlling the other one of the first control wire 110-1 and the second control wire 110-2 to also move toward the distal end, wherein a moving distance of the other one is less than a moving distance of the one of the first control wire 110-1 and the second control wire 110-2. More description regarding the clip instrument 10 switching to the side-open state may be found in the related description of FIG. 13-FIG. 15 above.

In some embodiments, controlling the clip instrument 10 to simultaneously clamp the first target object and the second target object includes: controlling, among the distal ends of the first control wire 110-1 and the second control wire 110-2, the one that is more distal to move toward the proximal end, thereby closing the clip instrument 10 to and achieve clamping. In some embodiments, controlling the clip instrument 10 to simultaneously clamp the first target object and the second target object further includes: controlling, among the distal ends of the first control wire 110-1 and the second control wire 110-2, the one that is more proximal to move toward the proximal end, thereby closing the clip instrument 10 and achieve clamping. More description may be found in the related description of FIG. 13 to FIG. 15 above.

In some embodiments, the clip base 320 includes the limiting portion 323. The limiting portion 323 is located between the first clamping portion 310-1 and the second clamping portion 310-2. The first clamping portion 310-1 and the second clamping portion 310-2 respectively include the stop portion 312. More description regarding the stop portion 312 may be found in the related description of FIG. 2-FIG. 4. In some embodiments, controlling the clip instrument 10 to switch to the side-open state further includes: controlling the first clamping portion 310-1 or the second clamping portion 310-2 deflects toward the limiting portion 323 to a preset position, the stop portion 312 contacts the limiting portion 323 to stop. In some embodiments, the preset position includes a position of the central axis B-B of the clip base 320. When the clamping portion 310 deflects in the direction D1 toward the central axis B-B of the clip base 320 to a position collinear with the central axis B-B of the clip base 320, the stop portion 312 contacts the limiting portion 323 to stop. In some embodiments, the preset position includes a position forming a preset angle with the central axis B-B of the clip base 320. The preset angle is within a range of 0 to 30 degrees. For example, the preset angle is 10 degrees. When the clamping portion 310 deflects in the direction D1 toward the central axis B-B of the clip base 320 to a position forming a 10-degree angle with the central axis B-B of the clip base 320, the stop portion 312 contacts the limiting portion 323 to stop.

In some embodiments, at least the other one of the first clamping portion 310-1 and the second clamping portion 310-2 includes the puncture platform 340. The puncture platform 340 includes the puncture space 341. More description regarding the puncture platform 340 may be found in the related description of FIG. 23-FIG. 24. In some embodiments, controlling the clip instrument 10 to clamp the first target object at the first target position and causing the puncture member 330 to penetrate into the first target object includes: controlling the puncture platform 340 or the puncture member 330 to abut against the first target object; controlling the puncture platform 340 and the puncture member 330 to approach each other until the puncture platform 340 and the puncture member 330 simultaneously abut against the first target object; controlling the clip instrument 10 to close, causing the puncture member 330 to pass through the first target object and pierce into the puncture space 341.

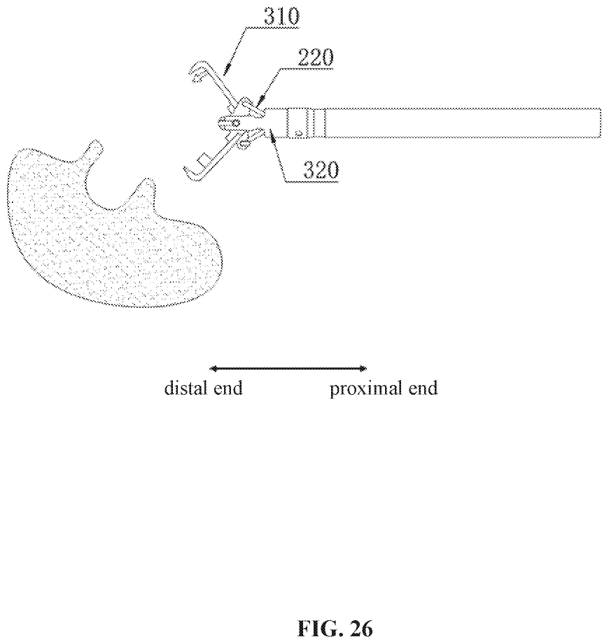

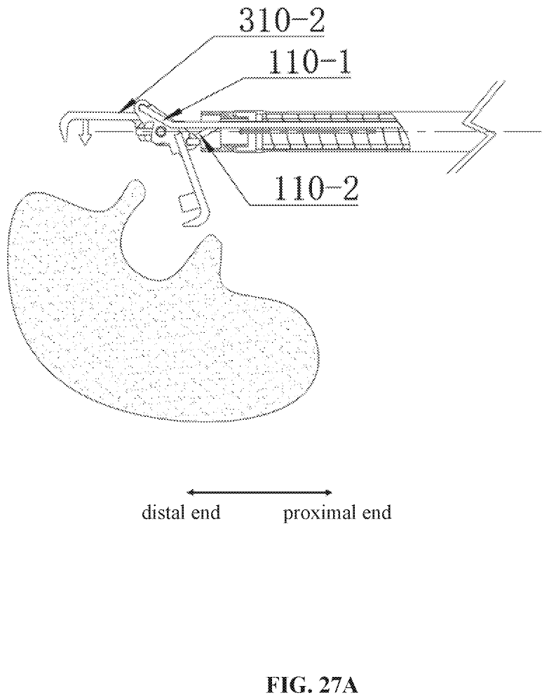

The operational process of the control method is described below with reference to FIG. 26 to FIG. 31B. Description regarding controlling deflection may be found in the aforementioned control method. The processes shown below are for illustrative purposes only. In some embodiments, the process may be performed utilizing one or more additional operations not described and/or without one or more of the operations discussed. Furthermore, the order of the operations of the process described below is not intended to be limiting.

FIG. 26 is a schematic diagram of the open state of the clip instrument 10 according to some other embodiments of the present disclosure. At this time, the clip instrument 10 does not clamp a tissue, and the clip instrument 10 is in the open state. In some embodiments, a span of the clip instrument 10 may be 10-24 mm. In some embodiments, the span of the clip instrument 10 may be 12-24 mm. In some embodiments, the span of the clip instrument 10 may be 12-22 mm. In some embodiments, the span of the clip instrument 10 may be 15-20 mm. In some embodiments, the span of the clip instrument 10 may be 17-20 mm. In some embodiments, the span of the clip instrument 10 may be 17.5-18.5 mm. In some embodiments, the span of the clip instrument 10 may be 18 mm. The span refers to a distance between distal ends of the two clamping portions.

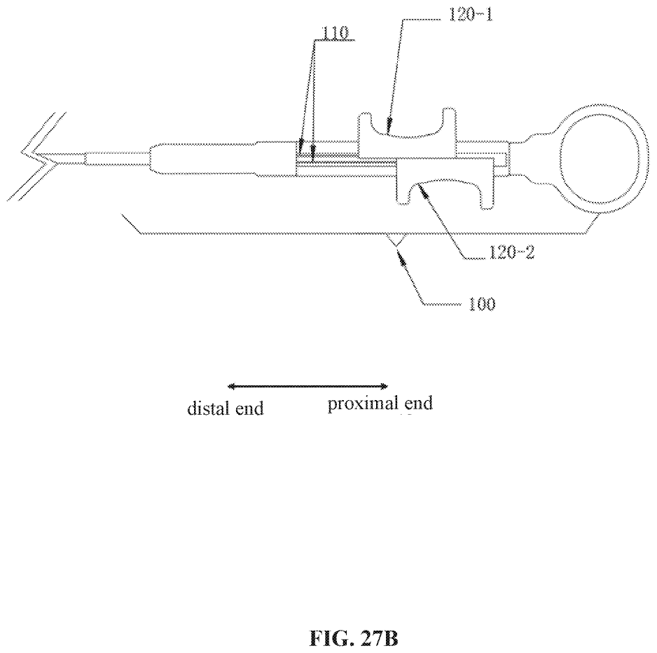

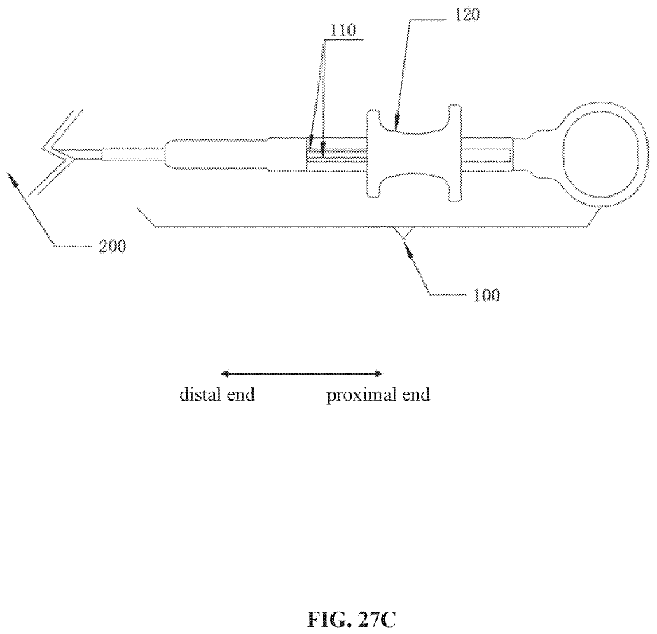

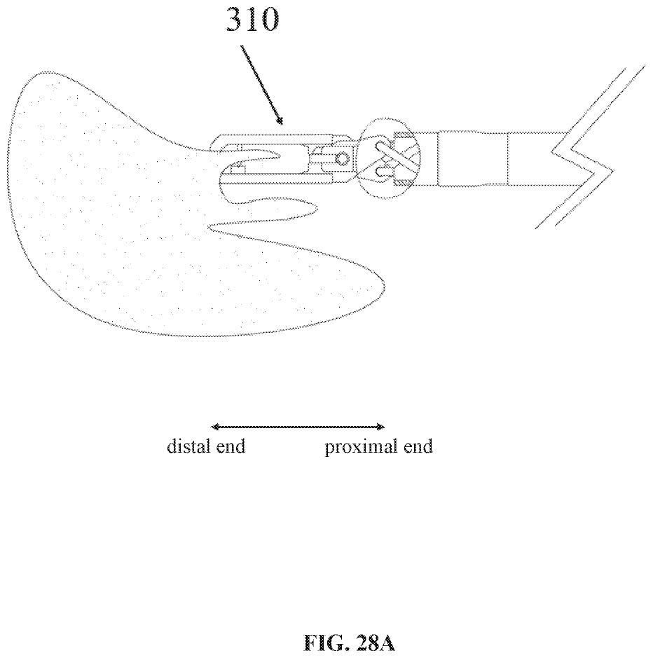

FIG. 27A is a schematic diagram of the deflected state of the clip instrument 10 according to some other embodiments of the present disclosure. FIG. 27B is a schematic diagram of a control handle in the deflected state according to some embodiments of FIG. 27A. As shown in FIG. 27B, when the first control handle 120-1 moves toward the distal end, the first control wire 110-1 is driven to move toward the distal end. Simultaneously, the second control handle 120-2 moves toward the proximal end, driving the second control wire 110-2 to move toward the proximal end. At this time, the first clamping portion 310-1 opens, and the deflection is achieved (see FIG. 27A). FIG. 27C is a schematic diagram of a control handle in the deflected state according to some other embodiments of FIG. 27A. As shown in FIG. 27C, when the control handle 120 moves toward the distal end, the clip instrument 10 opens (see FIG. 26). The first clamping portion 310 abuts against the tissue, and the tissue is clamped, causing the puncture member 330 to penetrate into the tissue. The clip instrument 10 is opened again, and the control device 100 is pushed toward the distal end, and the deflection is achieved (see FIG. 27A).

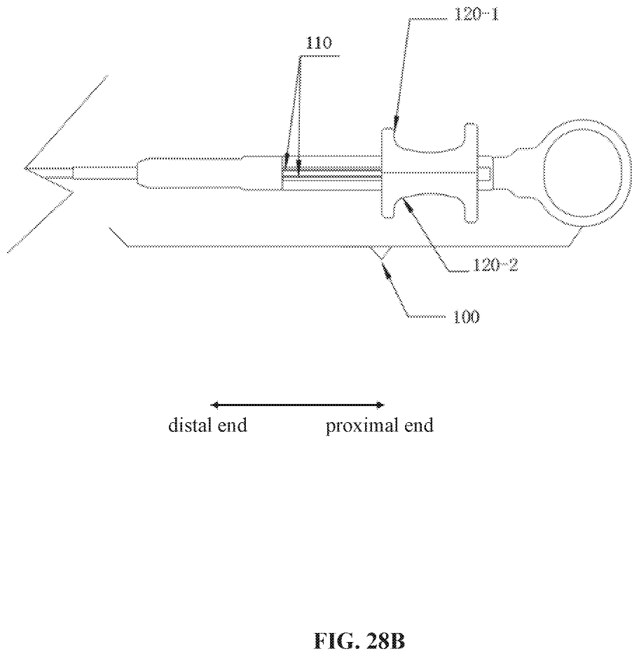

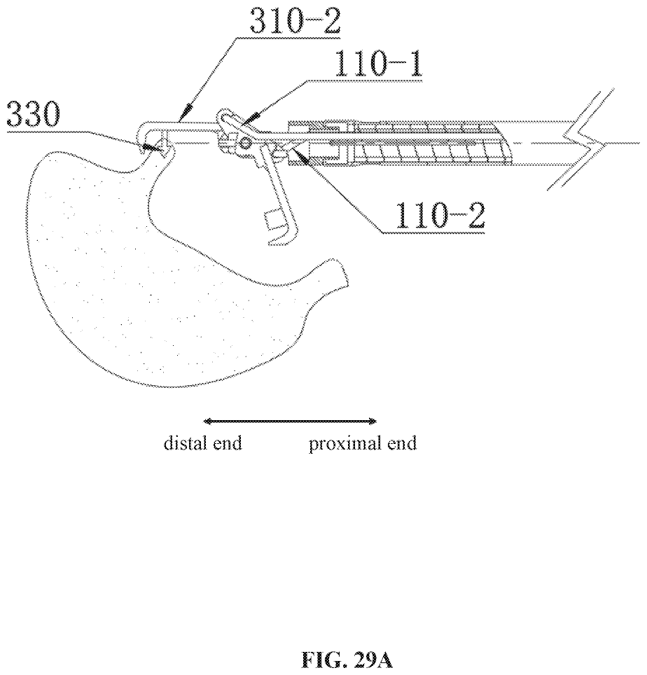

FIG. 28A is a schematic diagram of the clip instrument 10 clamping tissue after side-opening according to some embodiments of the present disclosure. FIG. 28B is a schematic diagram of a control handle in a state according to some embodiments of FIG. 28A. As shown in FIG. 28B, the first control handle 120-1 is moved toward the proximal end, and the clip instrument 10 achieves closure (see FIG. 28A).

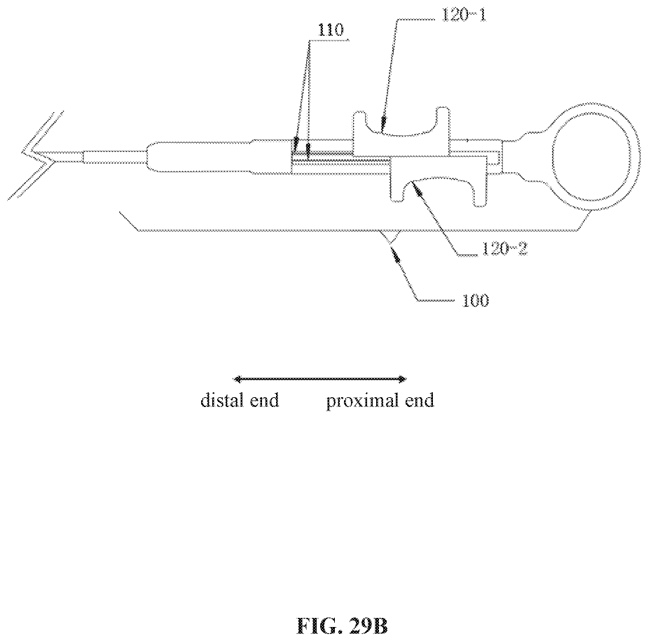

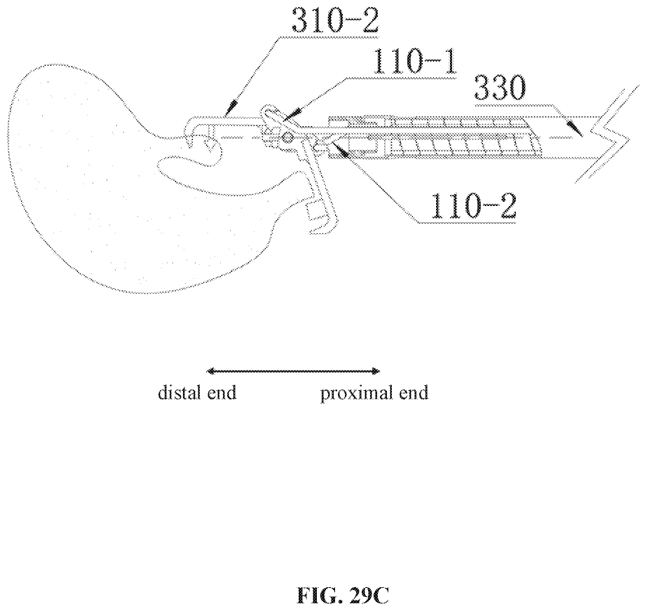

FIG. 29A is a schematic diagram of a secondary open state of the clip instrument 10 according to some embodiments of the present disclosure. FIG. 29B is a schematic diagram of a control handle in a state according to some embodiments of FIG. 29A. As shown in FIG. 29B, at least one control handle (e.g., the first control handle 120-1) is moved toward the distal end, and the clip instrument 10 reopens. At this time, the tissue is hooked on the puncture member 330 (see FIG. 28A).

FIG. 29C is a schematic diagram of a secondary open state of the clip instrument 10 according to some other embodiments of the present disclosure. As shown in FIG. 29C, when the tissue with a large span (e.g., larger than 18 mm) needs to be clamped, after the puncture member 330 penetrates into the tissue, the sheath 210 is pushed, causing the second clamping portion 310-2 to move the tissue toward the other side of the wound until both sides of the wound are within the space between the two clamping portions.

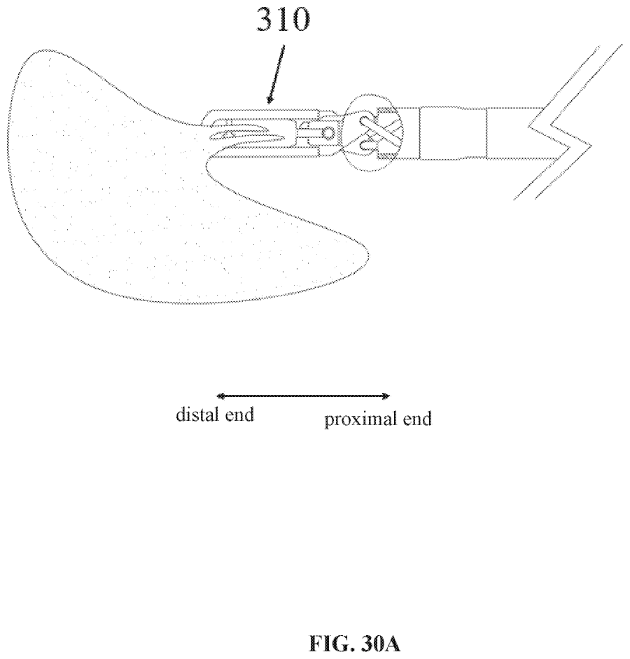

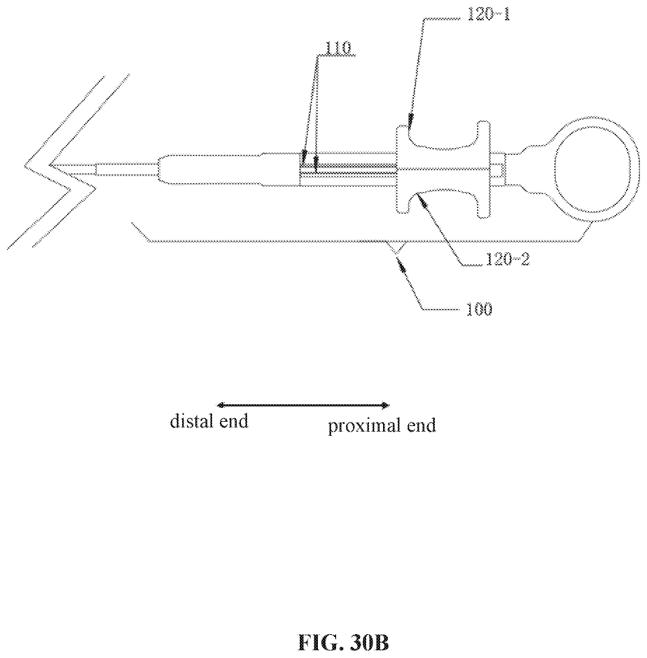

FIG. 30A is a schematic diagram of a secondary closed state of the clip instrument 10 according to some embodiments of the present disclosure. FIG. 30B is a schematic diagram of a control handle in a state according to some embodiments of FIG. 30A. As shown in FIG. 30B, moving the first control handle 120-1 toward the proximal end causes the clip instrument 10 to close, and two sides of the wound of the tissue are clamped by the clamping portion 310 (see FIG. 30A).

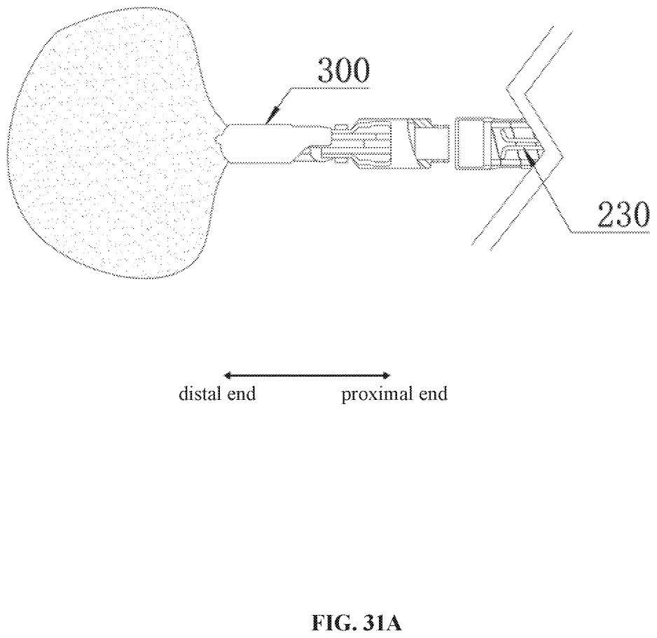

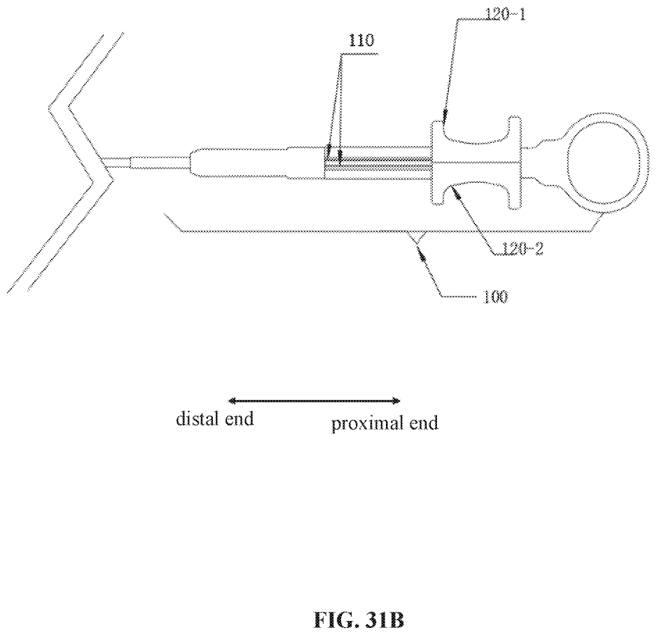

FIG. 31A is a schematic diagram of a released state of the clip device 300 and the delivery device 200 according to some other embodiments of the present disclosure. FIG. 31B is a schematic diagram of a control handle in a state according to some embodiments of FIG. 31A. As shown in FIG. 31B, moving both the first control handle 120-1 and the second control handle 120-2 toward the proximal end causes the clip device 300 to be released and separated from the delivery device 200 (see FIG. 31A).

Possible beneficial effects of embodiments of the present disclosure include, but are not limited to the following.

(1) In clinical operations, after the two clamping portions are opened, the clamping portions are operated to deflect in the same direction toward a wound of tissue, which facilitates effective closure of the wound of the tissue, ensuring a closure effect. Furthermore, after the clamping portions are operated to be open, an operator can operate the clamping portions to rotate through the control device without changing the control handle, which is more convenient and simpler than operating a distal end of an endoscope to rotate by changing an endoscope handle, thereby saving surgical time and reducing surgical risks.

(2) The limiting portion of the clip base limits a deflection angle of the clamping portion. When two clamping portions deflect in the same direction, the limiting portion can prevent the deflection angle of the clamping portion from being too large and exceeding a target deflection angle range, thereby avoiding a situation where the clamping portion cannot accurately face the wound of the tissue. Furthermore, the limiting portion limits the clamping portion to deflect within a specific angle range, thereby avoiding accidents such as scratching an endoscope channel or other unexpected accidents.

(3) Controlling the deflection of the first clamping portion through the first control wire and the deflection of the second clamping portion through the second control wire is simple and fast. Furthermore, movement between the first clamping portion and the second clamping portion does not cause interference. Therefore, the operator can control the first clamping portion and the second clamping portion to deflect sequentially or synchronously according to actual situations, making the operation more flexible.

(4) By deflecting the first clamping portion by applying a force to the first clamping portion with an external object and causing the second clamping portion to deflect through deflection of the first clamping portion, the need for the operator to operate the first clamping portion and the second clamping portion is eliminated. The operator only needs to adjust the endoscope to achieve the deflection of the two clamping portions, thereby further simplifying the operation. Furthermore, the first clamping portion can abut against an edge of the wound and then deflect, which can more accurately orient a clamping space of the clamping portion toward the wound, achieving precise positioning.

(5) The operator controls the two control wires separately through the first control handle and the second control handle, making the movement of the two control wires independent. The two control wires further control the two clamping portions separately, ensuring that the deflection of the two clamping portions does not interfere with each other. Therefore, operations such as opening, integral deflection, locking, closing, and releasing can be performed, making the operation more flexible.

(6) The clip base is rotatably connected to the sheath. The rotation angle of the clip base can be adjusted to align the closed direction of the two clamping portions with the closed direction of the wound of the tissue, thereby effectively achieving closure of the wound of the tissue.

(7) Providing the puncture member with the hooking portion can stably clamp the tissue between the clamping portions, improving clamping stability of the clip instrument and preventing tissue slippage.

(8) Combining the puncture member with the deflectable clip portion makes clamping of the clip instrument more flexible and enables clamping over a large span.

It should be noted that different embodiments may produce different beneficial effects. In different embodiments, the beneficial effects produced may be any one or a combination of the above or may be any other possible beneficial effects that can be obtained.

The foregoing has described basic concepts. Obviously, for those skilled in the art, the foregoing detailed disclosure is merely an example and does not constitute a limitation on the present disclosure. Although not explicitly stated herein, those skilled in the art may make various modifications, improvements, and corrections to the present disclosure. Such modifications, improvements, and corrections are suggested in the present disclosure. Therefore, such modifications, improvements, and corrections still fall within the spirit and scope of the exemplary embodiments of the present disclosure.

Meanwhile, the present disclosure uses specific words to describe the embodiments of the present disclosure. For example, “an embodiment,” “one embodiment,” and/or “some embodiments” mean that a certain feature, structure, or characteristic is related to at least one embodiment of the present disclosure. Therefore, it should be emphasized and noted that “an embodiment” or “one embodiment” or “an alternative embodiment” mentioned two or more times in different places in the present disclosure does not necessarily refer to the same embodiment. In addition, certain features, structures, or characteristics in one or more embodiments of the present disclosure may be appropriately combined.

Similarly, it should be noted that, in order to simplify the expression disclosed in the present disclosure and thereby help understand one or more inventive embodiments, in the foregoing description of the embodiments of the present disclosure, various features are sometimes grouped into one embodiment, drawing, or description thereof. However, this disclosure method does not mean that the object of the present disclosure requires more features than those mentioned in the claims. Rather, claimed subject matter may lie in less than all features of a single foregoing disclosed embodiment.

In some embodiments, numbers describing quantities of components or attributes are used. It should be understood that such numbers used to describe the embodiments are, in some examples, modified by the modifiers “about,” “approximately,” or “substantially.” Unless otherwise stated, “about,” “approximately,” or “substantially” indicates that the stated number allows a variation of ±20%. Accordingly, in some embodiments, numerical parameters used in the specification and claims are approximate values. The approximate values may vary according to characteristics required by individual embodiments. In some embodiments, numerical parameters should consider specified significant digits and adopt a general manner of digit retention. Although numerical ranges and parameters used to confirm the breadth of their ranges in some embodiments of the present disclosure are approximate values, in specific embodiments, setting of such numerical values is as precise as possible within a feasible range.

Finally, it should be understood that the embodiments described in the present disclosure are only used to illustrate principles of the embodiments of the present disclosure. Other variations may also fall within the scope of the present disclosure. Therefore, by way of example and not limitation, alternative configurations of the embodiments of the present disclosure may be considered consistent with the teachings of the present disclosure. Accordingly, the embodiments of the present disclosure are not limited to the embodiments explicitly introduced and described in the present disclosure.

What is claimed is:

1. A clip instrument, comprising: a clip device, a delivery device, and a control device, wherein:
the clip device is releasably disposed at a distal end of the delivery device, the control device includes a control wire, and the control wire includes a first control wire and a second control wire;
the clip device includes a clip base and a clamping portion, the clamping portion includes a first clamping portion and a second clamping portion, a distal end of the first control wire is releasably connected to the first clamping portion, and a distal end of the second control wire is releasably connected to the second clamping portion; and
the first clamping portion and/or the second clamping portion are configured to deflect in the same direction around an axis under an action of the first control wire and/or the second control wire, and the axis is perpendicular to a plane in which the first clamping portion and the second clamping portion are located.

2. The clip instrument of claim 1, wherein the clip base includes a limiting portion, the limiting portion is configured to limit a deflection angle of the first clamping portion and/or a deflection angle of the second clamping portion.

3. The clip instrument of claim 2, wherein the limiting portion is located between the first clamping portion and the second clamping portion, the first clamping portion and the second clamping portion respectively include a stop portion, when the first clamping portion or the second clamping portion deflects in a direction toward the limiting portion to a preset position, the stop portion contacts the limiting portion to stop.

4. The clip instrument of claim 1, wherein when the first control wire pushes the first clamping portion to deflect in a direction away from a central axis of the clip base, and the second control wire pulls the second clamping portion to deflect in a direction toward the central axis of the clip base, the first clamping portion and the second clamping portion deflect in the same direction around the axis.

5. The clip instrument of claim 1, wherein when a distal end of the first clamping portion is subjected to an external force, the first clamping portion deflects in a direction away from a central axis of the clip base, which drives the second clamping portion to deflect in a direction toward the central axis of the clip base, while simultaneously causing deformation of the second control wire, and the first clamping portion and the second clamping portion deflect in the same direction around the axis.

6. The clip instrument of claim 5, wherein the first clamping portion deflects in the direction away from the central axis of the clip base and abuts against the second control wire or the second clamping portion, which drives the second clamping portion to deflect in the direction toward the central axis of the clip base, while simultaneously causing the deformation of the second control wire.

7. The clip instrument of claim 6, wherein after the first clamping portion abuts against the second control wire or the second clamping portion, an included angle between the first clamping portion and the second clamping portion remains unchanged.

8. The clip instrument of claim 1, wherein the control device includes a first control handle and a second control handle, the first control handle is connected to the first control wire, the second control handle is connected to the second control wire, the first control handle controls the first clamping portion to deflect about the axis through the first control wire, and the second control handle controls the second clamping portion to deflect about the axis through the second control wire.

9. The clip instrument of claim 1, wherein the control device includes a control handle, the control handle is connected to the first control wire and the second control wire, the control handle simultaneously controls the first clamping portion and the second clamping portion to deflect around the axis by controlling the first control wire and the second control wire.

10. The clip instrument of claim 1, wherein the clip instrument includes an open state, the open state includes a straight-open state and a side-open state;
when the first control wire and/or the second control wire act on the first clamping portion and/or the second clamping portion in a first preset mode, the clip instrument switches from the straight-open state to the side-open state; and
the first preset mode includes: one of the first control wire and the second control wire moving toward the distal end.

11. The clip instrument of claim 10, wherein the clip instrument further includes a closed state;
when the first control wire and/or the second control wire act on the first clamping portion and/or the second clamping portion in a third preset mode, the clip instrument switches from the side-open state to the closed state; and
the third preset mode includes: among the distal end of the first control wire and the distal end of the second control wire, the one that is more distal moving toward a proximal end.

12. The clip instrument of claim 1, wherein one of the first clamping portion and the second clamping portion includes a locking portion, and the other of the first clamping portion and the second clamping portion includes a locked portion, the locking portion includes a locked state and a released state, when the clip device is connected to the control wire, the locking portion and the locked portion are capable of relative movement, and the locking portion is in the released state, when a connection between the clip device and the control wire is released, the locking portion and the locked portion are in a snap-fit connection, and the locking portion is in the locked state.

13. The clip instrument of claim 12, wherein the locking portion includes a locking space for accommodating the locked portion;
when the clip device is connected to the control wire, an opening size of the locking space is greater than a size of the locked portion; and
when the connection between the clip device and the control wire is released, the opening size of the locking space is less than the size of the locked portion, and the locked portion is snap-fitted in the locking space.

14. The clip instrument of claim 1, wherein at least one of the first clamping portion or the second clamping portion includes a puncture member.

15. A clip instrument, comprising: a clip device, wherein:
the clip device includes a clip base and a clamping portion, the clamping portion includes a first clamping portion and a second clamping portion, at least one of the first clamping portion or the second clamping portion includes a puncture member;
the clip instrument further comprises a delivery device and a control device, the clip device is releasably disposed at a distal end of the delivery device, the control device includes a control wire, the control wire includes a first control wire and a second control wire, a distal end of the first control wire is releasably connected to the first clamping portion, and a distal end of the second control wire is releasably connected to the second clamping portion.

16. The clip instrument of claim 15, wherein the first clamping portion includes a first clip arm and a first clip head connected to each other, a free end of the first clip head extends radially inward of the clip device, the puncture member is disposed on the first clip arm, and a ratio of a height of the puncture member to a height of the first clip head ranges from 1.2 to 3; and/or
the second clamping portion includes a second clip arm and a second clip head connected to each other, a free end of the second clip head extends radially inward of the clip device, the puncture member is disposed on the second clip arm, and a ratio of a height of the puncture member to a height of the second clip head ranges from 1.2 to 3.

17. The clip instrument of claim 15, wherein the puncture member is disposed close to an axis of the clamping portion, or the puncture member is disposed on a side of the clamping portion.

18. The clip instrument of claim 15, wherein at least the other of the first clamping portion and the second clamping portion includes a puncture platform, and the puncture platform is provided with a puncture space;
when the first clamping portion and the second clamping portion close, the puncture member penetrates into the puncture space; and
when the first clamping portion and the second clamping portion open, the puncture member withdraws from the puncture space.

19. A control method for a clip instrument, wherein:
the clip instrument includes a clip device, a delivery device, and a control device, the clip device is releasably disposed at a distal end of the delivery device, the control device includes a control wire, the control wire includes a first control wire and a second control wire, the clip device includes a clip base and a clamping portion, the clamping portion includes a first clamping portion and a second clamping portion, a distal end of the first control wire is releasably connected to the first clamping portion, and a distal end of the second control wire is releasably connected to the second clamping portion;
the control method comprises:
controlling the first control wire to move from a proximal end to a distal end of the first clamping portion by the control device to cause the first control wire to drive the first clamping portion to deflect around an axis; and
controlling the second control wire to move from a distal end to a proximal end of the second clamping portion by the control device to cause the second control wire to drive the second clamping portion to deflect around the axis;
wherein a deflection direction of the first clamping portion and a deflection direction of the second clamping portion are the same, and the clip device deflects integrally.

20. A control method for a clip instrument, wherein:
the clip instrument includes a clip device, a delivery device, and a control device, the clip device is releasably disposed at a distal end of the delivery device, the control device includes a control wire, the control wire includes the first control wire and the second control wire, the clip device includes a clip base and a clamping portion, the clamping portion includes a first clamping portion and a second clamping portion, a distal end of the first control wire is releasably connected to the first clamping portion, a distal end of the second control wire is releasably connected to the second clamping portion, and at least one of the first clamping portion or the second clamping portion includes a puncture member;
the control method comprises:
controlling the clip instrument to clamp a first target object at a first target position and causing the puncture member to penetrate into the first target object;
controlling at least a portion of the clip device to move to a second target position, wherein the puncture member drives the first target object to move synchronously; and
controlling the clip instrument to simultaneously clamp the first target object and a second target object.
